(19) United States
(12) Patent Application Publication
(10) Pub. No.: US 2010/0178268 A1
(43) Pub. Date: Jul. 15, 2010

(54) BIOCIDIC PACKAGING FOR COSMETICS AND FOODSTUFFS

(75) Inventors: Shmuel Bukshpan, Ramat Ha-Sharon (IL); Gleb Zilberstein, Rechovot (IL)

Correspondence Address: The Law Office of Michael E. Kondoudis, 888 16th Street, N.W., Suite 800, Washington, DC, 20006, US

(73) Assignee: OPLON B.V., Delft (NL)

(21) Appl. No.: 12/598,433

(22) PCT Filed: Apr. 3, 2008
(86) PCT No.: PCT/IL08/00468

§ 371 Date: Mar. 5, 2010

Related U.S. Application Data
(60) Provisional application No. 60/924,151, filed on May 1, 2007.
(60) Provisional application No. 60/924,146, filed on May 1, 2007.

(51) Int. Cl. A61K 31/74

(52) U.S. Cl. 424/78.08

(57) ABSTRACT

The present invention presents a biocidic packaging for cosmetics and/or foodstuffs, comprises at least one insoluble proton sink or source (PSS). The packaging is provided useful for killing living target cells (LTCs), or otherwise disrupting vital intracellular processes and/or intercellular interactions of said LTC upon contact. The PSS comprises, inter alia, (i) proton source or sink providing a buffering capacity; and (ii) means providing proton conductivity and/or electrical potential. The PSS is effectively disrupting the pH homeostasis and/or electrical balance within the confined volume of the LTC and/or disrupting vital intercellular interactions of the LTCs while efficiently preserving the pH of said LTCs' environment. The present invention also discloses a method for killing living target cells (LTCs), or otherwise disrupting vital intracellular processes and/or intercellular interactions of said LTC being in a packaging, especially cosmetic or foodstuffs' packaging.

### FIELD OF THE INVENTION

The present invention pertains to biocidic packaging for cosmetics and foodstuffs. The present invention also relates to a method for avoiding contamination of cosmetics and food stuffs in their packaging.

### BACKGROUND OF THE INVENTION

In general cosmetics and food stuffs are easily contaminated by bacteria, fungi etc. To prevent this contamination most of the cosmetics and food stuffs formulations include preservatives necessary to prevent microbial contamination common in any use of cosmetics and food stuffs. Unfortunately most of the preservatives added to cosmetics are toxic and may be skin irritating or cause infection. Much similarly, it is a long felt need for the food industry to eliminate, or at least to decrease, the preservatives content in the food. For sack of clarification, the background will first focus on the cosmetics industry, and than will approach the food packaging industry.

### Cosmetics

A large variety of preservative materials have been utilized in the cosmetic industry. One of the eldest and most commonly used preservative by the industry for a long time are esters of para-hydroxybenzoic acid, collectively known as the parabens.

Due to the high toxicity of parabens, the cosmetic industry is in a continuous search for both (i) alternative preservation systems to the traditional paraben mixtures and (ii) various low toxicity combinations designed to enhance preservative efficacy.

Hence, a non-toxic and non-irritating biocide, which effectively destroys or inhibits growth of micro-organisms such as bacteria, yeasts and moulds, is still an unmet need.

A list of preservative materials compiled in 2005 is headed by methyl- and propyl-paraben and includes a limited number of preservative materials. A non complete list of other preservative materials commonly used in the industry is as follows: Imidazolidynyl urea, Phenoxyethanol, Formaldehyde, Quaternium 15, Methylchloroisothiazolinone, a synergistic blend of methylisothiazolinone and polyaminopropyl biguanide (MTB), a blend of methylisothiazolinone and chlorphenesin (MTC), a synergistic combination of methylisothiazolinone and iodopropynyl butylcarbamate (MTI), Iodopropynyl butylcarbamate (IPBC), Rockonsal ND a combination of benzoic acid and dehydroacetic acid in phenoxyethanol, Rokonsal BSB is a combination of benzoic and sorbic acids in benzyl alcohol, Australian myrtle oil, Usnic acid, JM ActiCare™, a suspension of particles of a silver chloride/titanium dioxide composite in a water/sulfosuccinate gel, Polyaminopropyl biguanide etc.

Preservatives in general and certain groups in particular, have had a bad press in the last years and some manufacturers have already chosen to reformulate. The industry is seeing a backlash against preservatives by significant numbers of consumers. Also, there is potential conflict between the need for non-contaminated products and their toxicological safety. Today, cosmetic products can only use a limited number of preservatives selected from a positive list, e.g., Annex VI of the Cosmetics Directive, which also defines their maximum permitted levels and areas of use EPC Directive 94/62/EC.

Facing consumers rebellion against preservatives in general and some in particular, and safety assessors questioning the inclusion of preservatives, even when incorporated according to the levels and practices of use laid down by the Cosmetics Directive there is a continuous need for innovative, safer and more acceptable alternative methods for preservation of cosmetics.

a. Foodstuffs

Packages have become an essential element in current developed societies. In particular, food packaging has experienced an extraordinary expansion, because most commercialized foodstuffs, including fresh fruits and vegetables, are being marketed inside packages. One important function of packaging, when regarded as a food preservation technology, is to retard food product deterioration, extending shelf-life, and to maintain and increase the quality and safety of the packaged foods. Thus, the main purpose of food packaging is to protect the food from microbial and chemical contamination, oxygen, water vapor, and light. The type of package used, therefore, has an important role in determining the shelf-life of food. By means of the correct selection of materials and packaging technologies, it is possible to keep the product quality and freshness during the period required for its commercialization and consumption.

Traditionally, food packages have been defined as passive barriers to delay the adverse effect of the environment on the contained product. However, the current tendencies include the development of packaging materials that interact with the environment and with the food, playing an active role in preservation. These new food packaging systems have been developed as a response to trends in consumer preferences toward mildly preserved, fresh, tasty, and convenient food products with a prolonged shelf-life. In addition, changes in retail practices, such as globalization of markets resulting in longer distribution distances, present major challenges to the food packaging industry acting as driving forces for the development of new and improved packaging concepts that extend shelf-life, while maintaining the safety and quality of the packaged food.

Active packaging refers to those technologies intended to interact with the internal gas environment and/or directly with the product, with a beneficial outcome. The first designs in active packaging made use of a small pouch (sachet) containing the active ingredient inserted inside the permeable package. This technology yields some attractive characteristics, especially a high activity rate and lack of complex equipment or modification of packaging procedures because the sachet is inserted in an additional step. However, there are many disadvantages related to the use of sachets, the most important one being the presence inside the package of substances that are often toxic and could be accidentally eaten or may cause consumer rejection.

The alternative, which is being extensively studied, is the incorporation of the active substance within the package material wall. Plastics are really convenient materials for this sort of technologies, not only as vehicles of the active substance, but also participating as active parts of the active principle. Hence, an important objective here is to design functional plastic materials that include the active agent in their structure and that this active substance can act or be released in a controlled manner. The additional advantages of incorporating this active agent in the polymeric structure (package wall) over their use in sachets are, for example, package size reduction, sometimes higher efficiency of the active substance (which is completely surrounding the product) and higher output in the packaging production (as the incorporation of the sachet means an additional step, generally manual). Some precautions and considerations have to be taken into account when applying these active plastics. The active agent may change the plastic properties, adsorption kinetics are variable and dependent on plastic permeability, the active capacity may get shortened by an early reaction if there is no effective triggering mechanism, and there is a potential undesired migration of active substances or low molecular weight reaction products into the food.

Most of the active agents are considered food-contact material constituents (instead of food additives), and therefore, these systems should comply with the very strict existing regulations regarding migration. Typical examples includes oxygen scavengers, carbon dioxide scavengers and emitters, ethylene scavengers, water absorbers and regulators, organic compound absorbers and emitters, enzymatically active films, and antimicrobial systems. Food Contact Materials are traditionally comprising flexible films, that usually have the following properties: Their cost is relatively low; They have good barrier properties against moisture and gases; They are heat sealable to prevent leakage of contents; They have wet and dry strength; They are easy to handle and convenient for the manufacturer, retailer and consumer; They add little weight to the product; They fit closely to the shape of the food, thereby wasting little space during storage and distribution etc.

A short summary of the different types of flexible films is as follows:

Cellulose Plain cellulose is a glossy transparent film which is odorless, tasteless and biodegradable (within approximately 100 days). It is tough and puncture resistant, although it tears easily. However, it is not heat sealable and the dimensions and permeability of the film vary with changes in humidity. It is used for foods that do not require a complete moisture or gas barrier.

Polypropylene Polypropylene is a clear glossy film with a high strength and is puncture resistance. It has moderate permeability to moisture, gases and odors, which is not affected by changes in humidity. It stretches, although less than polyethylene.

Polyethylene Low-density polyethylene is heat sealable, inert, odor free and shrinks when heated. It is a good moisture barrier but has relatively high gas permeability, sensitivity to oils and poor odor resistance. It is less expensive than most films and is therefore widely used. High-density polyethylene is stronger, thicker, less flexible and more brittle than low-density polyethylene and has lower permeability to gases and moisture. It has higher softening temperature (121° C.) and can therefore be heat sterilized. Sacks made from 0.03-0.15 mm high-density polyethylene have high tear strength, penetration resistance and seal strength. They are waterproof and chemically resistant and are used instead of paper sacks.

Other films Polystyrene is a brittle clear sparkling film which has high gas permeability. Polyvinylidene chloride is very strong and is therefore used in thin films. It has very low gas and water vapor permeability and is heat shrinkable and heat sealable. However, it has a brown tint which limits its use in some applications. Nylon has good mechanical properties a wide temperature range (from 60 to 200° C.). However, the films are expensive to produce, they require high temperatures to form a heat seal, and the permeability changes at different storage humidity.

Coated films Films are coated with other polymers or aluminum to improve the barrier properties or to import heat sealability. For example, nitrocellulose is coated on one side of cellulose film to provide a moisture barrier but to retain oxygen permeability. A nitrocellulose coating on both sides of the film improves the barrier to oxygen, moisture and odors and enables the film to be heat sealed when broad seals are used. A coating of vinyl chloride or vinyl acetate gives a stiffer film which has intermediate permeability. Sleeves of this material are tough, stretchable and permeable to air, smoke and moisture. They are used, for example, for packaging meats before smoking and cooking. A thin coating of aluminum produces a very good barrier to oils, gases, moisture, odors and light. The properties are shown in Table 1.

TABLE 1
Properties of selected packaging materials

| | | | | | | Normal |
|---|---|---|---|---|---|---|
| | | Barriers to | | | | Thickness |
| Film Type | Coating | Moisture | Air/Odors | Strength | Clarity | (□m) |
| Cellulose | — | * | *** | * | *** | 21-40 |
| Cellulose | PVDC | *** | *** | * | *** | 19-42 |
| Cellulose | Aluminum | *** | *** | * | — | 21-42 |
| Cellulose | Nitro- | *** | *** | * | — | 21-24 |
| | cellulose | | | | | |
| Polyethylene | — | ** | * | ** | * | 25-200 |
| (low density) | | | | | | |
| Polyethylene | — | *** | ** | *** | * | 350-1000 |
| (high density) | | | | | | |
| Polypropylene | — | *** | * | *** | *** | 20-40 |
| Polypropylene | PVDC | *** | *** | *** | *** | 18-34 |
| Polypropylene | Aluminum | *** | *** | *** | — | 20-30 |
| Polyester | | ** | ** | *** | ** | 12-23 |
| Polyester | | *** | *** | *** | ** | — |
| Polyester | | *** | *** | *** | — | 20-30 |

* = low
** = medium
*** = high.
Thicker films of each type have better barrier properties than thinner films.
PVDC = polyvinylidehe chloride.

Laminated films Lamination of two or more films improves the appearance, barrier properties or mechanical strength of a package.

Co-extruded films This is the simultaneous extrusion of two or more layers of different polymers. Co-extruded films have three main advantages over other types of film: They have very high barrier properties, similar to laminates but produced at a lower cost; They are thinner than laminates and are therefore easier to use on filling equipment; The layers do not separate etc.

Examples of the use of laminated and co-extruded films are as follows:

TABLE 2
Selected laminated films used for food packaging

| Type of laminate | Typical food application |
|---|---|
| Polyvinylidene chloride coated | Crisps, snack foods, confectionery, |
| polypropylene (2 layers) | ice cream, biscuits, chocolate |
| Polyvinylidene chloride coated | Bakery products, cheese, |
| polypropylene-polyethylene | confectionery, dried fruit, |
| | frozen vegetables |
| Cellulose-polyethylene-cellulose | Pies, crusty bread, bacon, coffee, |
| | cooked meats, cheese |
| Cellulose-acetate-paper-foil- | Dried soups |
| polyethylene | |
| Metalized polyester-polyethylene | Coffee, dried milk |
| Polyethylene-aluminum-paper | Dried soup, dried vegetables, |
| | chocolate |

TABLE 3
Selected applications of co-extruded films

| Type of co-extrusion | Application |
|---|---|
| High impact polystyrene- | Margarine, butter tubs |
| polyethylene terephthalate | |
| Polystyrene-polystyrene- | Juices, milk bottles |
| polyvinylidene chloride-polystyrene | |
| Polystyrene-polystyrene- | Butter, cheese, margarine, coffee, |
| polyvinylidene chloride-polyethylene | mayonnaise, sauce tubs and bottles |

With the increasing use of polymeric materials for construction of medical apparatuses and packaging and handling of food products, utilizing an antimicrobial polymer has become ever more desirable.

Anti-Microbial Food Packaging Research into the area of antimicrobial food packaging materials has increased significantly during the past 10 years (Cooksey, 2001) as an alternative method to control undesirable microorganisms on foods by means of the incorporation of antimicrobial substances in or coated onto the packaging materials (Han, 2000). Because microbial contamination of most foods occurs primarily at the surface, due to post processing handling, attempts have been made to improve safety and delay spoilage by using antibacterial sprays or dips. However, direct surface application of antimicrobial substances has limited benefits because the active substances are neutralized or diffuse rapidly from the surface into the food mass. Therefore, the use of packaging films containing antimicrobial agents could be more efficient if high concentrations are maintained where they are needed by slow migration or action of the agents onto the surface of the product (Quintavalla and Vicini, 2002).

The major potential food applications for antimicrobial films include meat, fish, poultry, bread, cheese, fruits, vegetables, and beverages (Labuza and Breene, 1989).

Nowadays, antimicrobial food packaging is based on one of the following concepts: The package is designed to modify the environmental conditions inhibiting microbial growth. Previously described oxygen scavengers or CO₂ emitters alter the atmospheric composition and reduce the growth kinetics of aerobic microorganisms. Also, active packages that reduce water content affect microbial development. Some absorbing pads (diapers), used to soak up the exudates in meat trays, incorporate organic acids and surfactants in order to prevent microbial growth, because the food exudates are rich in nutrients (Hansen et al., 1988).

The package incorporates antimicrobial agents and is designed to release them into the headspace of the package or directly into the food product.

The package contains an immobilized substance with antimicrobial character. This category of active packages includes (i) polymers with inherent antimicrobial properties and (ii) structures that contain immobilized antimicrobial agents. Immobilization can be achieved by restricted diffusion or by covalent bonding of the substance to the polymer backbone. Although, currently, there are only a few food-related commercial applications of these technologies, this is an area of great interest and many research efforts are focused on their development and implementation.

For those antimicrobial substances that are to be released from the films, mass transfer is a critical issue to be considered in the design of the active system. The studies carried out on migration of volatile and nonvolatile organic molecules from polymers are applicable to describe the release of antimicrobial agents from packages (Garde et al., 2001; Katan, 1996). For volatile agents, their release is mainly controlled by their diffusion through the polymer and their vapor partial pressure at saturation. Once in the headspace, antimicrobial substances reach the surface of the food where they are adsorbed and then dispersed or diffused throughout the food product.

Antimicrobial release from polymers has to be maintained at an adequate rate so the surface concentration is above a critical inhibitory concentration. To achieve appropriate controlled release to the food surface, the use of multilayer films (control layer/matrix layer/barrier layer) has been proposed. The inner layer controls the rate of diffusion of the active substance, whereas the matrix layer contains the active substance and the barrier layer prevents migration of the agent toward the outside of the package (Cooksey, 2001).

Many volatile compounds are known to exhibit antimicrobial properties, including gases, such as SO₂ or ClO₂, and vapors of diverse volatility, including alcohols, aldehydes, ketones, and esters. Chlorine dioxide has received Food and Drug Administration acceptance as an antimicrobial additive for packaging materials. It is an antimicrobial gas released from a basic chlorine-containing chemical upon exposure to moisture. Its main advantage is that it functions at a distance and thus is one of the few packaging antimicrobials that do not require direct contact with the food.

Although testing results indicate efficacy in retarding mold growth on berries, results with fresh red meat are overshadowed by serious adverse color changes (Brody, 2001). CSIRO (Australia) is developing systems that gradually release SO₂ to control mold growth in some fruits. This application is not allowed in the European Union and it is important to remark that the accumulation or absorption of large quantities of SO₂ by foods could cause toxicological problems (Vermeiren et al., 2002).

There is currently active research focused on the isolation of natural compounds from foods and plants with fungicidal and bactericidal activity. The purpose of these studies was to obtain active packaging systems that combine modified atmosphere packaging with a controlled release of the active compound. The step to introduce these highly volatile compounds in the package wall is not simple because the film manufacturing process (solution casting or extrusion) results in the volatilization of the compound and a nonbreathable atmosphere in the production plant. A possible solution to this problem consists of using compounds that trap the active molecules and decrease their volatility. Cyclodextrin complexes have been used for these purposes, preserving flavors during extrusion processes (Bhandari et al., 2001; Reineccius et al., 2002). Some antimicrobial agents, flavor essences, horseradish essences, and ethanol have been successfully encapsulated in cyclodextrins (Ikushima et al., 2002).

Other less volatile natural compounds obtained from plants, including several fatty acids and essential oils, have been examined against various spoilage organisms. For nonvolatile compounds, direct contact between the package and the food surface is needed. Although diffusion of these compounds within the package walls affects their release, the type and state of food and the type of contact is also critical. Nonvolatile antimicrobial substances include some food preservatives such as sorbates, benzoates, propionates, and parabens, all of which are covered by U.S. FDA regulations (Floros et al., 1997). Sorbate-releasing plastic films are used for cheese packaging. Ionomer film with benzoyl chloride that showed potential as antimicrobial film through the release of benzoic acid to a buffer solution or to a potato dextrose agar media was also developed. Films containing sodium propionate have also been proved to be useful in prolonging the shelf-life of bread by retarding microbial growth (Soares et al., 2002).

An interesting commercial development is the more recent commercialization of food contact approved Microban® (Microban Products Co., USA) kitchen products, such as chopping boards or dish cloths that contain triclosan, an antimicrobial aromatic chloroorganic compound that is also used in soaps, and shampoos (Berenzon and Saguy, 1998). More recently, the use of triclosan for food-contact applications has been allowed in EU countries, with a maximum specific migration limit of 5 mg/kg of food (Quintavalla and Vicini, 2002). Vermeiren et al. (2002) demonstrated that the incorporation of triclosan into a low-density polyethylene resulted in activity in plate overlay assays, but when the plastic was combined with vacuum packaging and refrigerated storage, bacteria were not sufficiently reduced on meat surfaces. The possible interaction of triclosan with adipose components of the meat product may be responsible for this inactivity. Chung et al. (2001a, 2001b) studied the release of triclosan from a styrene-acrylate copolymer into water and fatty food simulants. In another study (Chung et al., 2003), a coating made of a styrene acrylate copolymer containing triclosans was seen to inhibit the growth of Enterococcus faecalis in agar diffusion tests, as well as in liquid culture tests. The data suggested that a styrene-acrylate copolymer containing triclosan could be an effective antimicrobial layers under appropriate conditions, although further research is needed to evaluate its effectiveness against other microorganisms.

Diverse enzymes and peptides have also been tested for their bactericidal capacity. Their low tolerance to temperature restricts the application of these compounds to their sorption into the polymer surface, or coating or casting from solutions. Lysozyme has been tested alone or in combination with plant extracts, nisin, or EDTA in various polymer films, including polyvinyl alcohol, polyamide, cellulose triacetate, alginate, and carrageenan films (Appendini and Hotchkiss, 1997; Buonocore et al., 2003; Cha et al., 2002). Other examples include nisin/methylcellulose coatings for polyethylene films (Cooksey, 2000), antimycotic agents incorporated into edible coatings from waxes and cellulose ethers (Hotchkiss, 1995), and nisin/zein coatings for poultry (http://www.uark.edu/depts/fsc/news.sum00.pdf (accessed October 2003)). Nisin, a bacteriocin produced by Lactococcus lactis, is considered to be a natural additive. It has GRAS (or “generally recognized as safe”) status for use with processed cheese, and it is particularly effective for preventing Clostridium botulinum growth (Cooksey, 2001). Recently, two different nisin-incorporated coatings (one with a binder solution of acrylic polymer and the other with a vinyl acetateethylene copolymer) have been studied for their antimicrobial activity, and when they were in contact with pasteurized milk and orange juice at 10° C., significant suppression of total aerobic bacteria and yeasts was observed (Kim et al., 2002).

Besides antimicrobial agents, which are released to exert a positive effect on the food product, some substances are completely immobilized in the package wall, and therefore, they only protect from microbial spoilage by direct contact with food surface. Focusing on this type of antimicrobial polymers, silver (Ag)-substituted zeolite is the most common antimicrobial agent incorporated into plastics commercialized in Japan (Vermeiren et al., 1999). Ag-ions that inhibit a range of metabolic enzymes have strong antimicrobial activity. Takayama et al. (1994) and Wirtanen et al. (2001) studied their efficacy on diverse microorganisms, including Pseudomonas, Bacillus, Staphylococcus, Micrococcus, enterobacteria and yeasts, reporting the broad antimicrobial spectrum of Ag-zeolites and their efficiency at low concentration (Kim and Lee, 2002). However, because it is expensive, Ag-zeolite is laminated as a thin layer (3-6 mm) with normal incorporation level from 1% to 3% (http://pffc-online.com/ar/paper_active_packaging/ (accessed January 2004). However, the real effectiveness of this system has not been evaluated because the requisite migration from polymers is minimal and silver ion's antimicrobial effects are weakened by sulfur-containing amino acids in many food products (Brody, 2001). The most practical application of this system seems to be for low-nutrient beverages, such as tea or mineral water. Commercial examples of Ag-zeolites are Zeomic_ (Shinanen New Ceramics Co. Ltd., Japan), AgIon™ (AgIon Technologies Inc., USA), and Apacider_ (Sangi Group America, USA). More recently, Renaissance Chemicals Ltd. and Addmaster (UK) have obtained FDA and BGVv (German Federal Institute for Risk Assessment) approvals for a silver-ion based coating (JAMC™) in food-contact applications (Paper Preservation/Paper Biocide, 2003).

Another way to immobilize antimicrobial substances is by ionic or covalent linkages to polymers. This type of immobilization requires the presence of functional groups on both the antimicrobial and the polymer. Examples of antimicrobials with functional groups are peptides, enzymes, polyamines, and organic acids. In addition, the use of “spacer” molecules that link the polymer surface with the BioActivity™ agent may also be required. Spacers that could potentially be used for food antimicrobial packaging include dextrans, polyethylene glycol, ethylenediamine and polyethyleneimine, due to their low toxicity and common use in foods (Appendini and Hotchkiss, 2002). Nisin and lacticin has been successfully attached to LDPE by using a polyamide binder (An et al., 2000; Kim et al., 2002).

Some polymers are inherently antimicrobial. Cationic polymers, such as chitosan and poly-L-lysine, promote cell adhesion, because charged amines interact with negative charges on the cell membrane, causing leakage of intracellular constituents. Chitosan is an aminopolysaccharide prepared by deacetylation of chitin, which is one of the most abundant natural polymers in living organisms such as crustaceans, insects and fungi. It has been proved to be nontoxic, biodegradable, and biocompatible (Kim et al., 2003). Chitosan has been used as a coating and appears to protect fresh vegetables and fruits from fungal degradation (Cuq et al., 1995). These films are effective against Listeria in cheese, although their antimicrobial activity decreases with time (Coma et al., 2002). Outtara et al. (2000a; 2000b) studied the synergistic effect of chitosan with diverse organic acids and cinammaldehyde. They found that all formulations were effective against various endogenous microorganisms in meat except for lactic acid bacteria, with the films with aldehyde presenting the highest efficiency. The greatest limitation of chitosan as a film material is its relatively poor mechanical properties. By crosslinking

chitosan films with dialdehyde starch, their mechanical properties are significantly improved and the films still retained obvious antimicrobial effects toward S. aureus and E. coli (Tang et al., 2003).

Another possibility to obtain antimicrobial polymers is by modifying their surfaces by introducing active functional groups. A novel method has been developed using a UV excimer laser. Nylon (6,6) films irradiated using a UV excimer laser at 193 nm in air possess antimicrobial activity, which results from the conversion of amide groups at the nylon surface to amines (with bactericidal properties) that are still bound in the polymer chain (Hagelstein et al., 1995). More recently, some antimicrobial polymers have been developed based on the application of porphyrin derivatives. These very large molecules are immobilized in a polymer film. The exposure of such film to light results in very reactive oxygen species. Singlet oxygen reacts with a broad variety of biomolecules becoming lethal for many microorganisms. These reactive oxygen molecules are released from the film and can present bactericidal activity in the food product. Currently, these materials are being used for medical textile fibres (Bozja et al., 2003; Sherrill et al., 2003), but their application to food packaging is still under study. A major concern of these films is their potential oxidative activity in foods, which can lead to rapid quality loss.

Antimicrobial packaging can play an important role in reducing the risk of pathogen contamination, as well as extending the shelf-life of foods. Probably, future work will focus on the use of biologically active derived antimicrobial compounds bound to polymers. The need for new antimicrobials with a wide spectrum of activity and low toxicity will increase. It is possible that research and development of antimicrobial packages will go beyond the current active packaging concept, giving rise to “intelligent” or “smart” packaging systems. These materials could be designed to perceive the presence of microorganisms in the food, triggering antimicrobial mechanisms (Appendini and Hotchkiss, 2002).

The following publications are hence incorporated as reference for the present invention: An, D. S., Kim, Y. M., Lee, S. B., Paik, H. D., Lee, D. S. (2000). Antimicrobial low density polyethylene film coated with bacteriocins in binder medium. Food Sci. Biotechnol. 9(1):14-20. Appendini, P., Hotchkiss, J. H. (2002). Review of antimicrobial food packaging. Innovative Food Sci. Emerging Technol. 3:113-126. Buonocore, G. G., Nobile, M. A., Panizza, A., Bove, S., Battaglia, G., Nicolais, L. (2003). Modeling the lysozyme release kinetics from antimicrobial films intended for food packaging applications. J. Food Sci. 68(4):1365-1370. Bhandari, B., D'Arcy, B., Young, G. (2001). Flavour retention during high temperature short time extrusion cooking process: a review. INTAL J. Food Sci. and Technol. 36(5):453-461. Berenzon, S., Saguy, I. S. (1998). Oxygen absorbers for extension of crakers shelflife. Food Sci. Technol. 31:1-5. Bozja, J., Sherrill, J., Michielsen, S., Stojiljkovic, I. (2003). Porphyrin-based, lightactivated antimicrobial materials. J. Polymer Sci. part A: Polymer Chem. 41:2297-2303. Brody, A. L. (2001). What's active in active packaging? Food Technol. 55:104-106. Cha, D. S., Choi, J. H., Chinnan, M. S., Park, H. J. (2002). Antimicrobial films based on Na alginate and Kappa-carrageenan. Lebensmittel-Wissenschaftund-Technologie 35(8):715-719. Chung, D., Chikindas, M. L., Yam, K. L. (2001a). Inhibition of Saccharomyces cerevisiae by slow release of propyl paraben from a polymer coating. J. Food Protection 64(9):1420-1424. Chung, D., Papadakis, S. E., Yam, K. L. (2001b). Release of propyl paraben from a polymer coating into water and food simulating solvents for antimicrobial packaging applications. J. Food Process. Preserv. 25(1):71-87. Chung, D., Papadakis, S. E., Yam, K. L. (2003). Evaluation of a polymer coating containing triclosan as the antimicrobial layer for packaging materials. Int. J. Food Sci. Technol. 38:165-169. Coma, V., Martial-Gros, A., Garreau, S., Copinet, A., Salin, F., Deschamps, A. (2002). Edible antimicrobial films based on chitosan matrix. J. Food Sci. 67(3):1162-1169. Cooksey, K. (2000). Utilization of antimicrobial packaging films for inhibition of selected microorganisms. In: Food Packaging: Testing Methods and Applications. Washington, D.C.: ACS. Cooksey, K. (2001). Antimicrobial food packaging materials. Additives for Polymers. pp. 6-10. Cuq, B., Gontard, N., Guilbert, S. (1995). Edible films and coatings as active layers. In: Rooney, M. L., ed. Active Food Packaging. London: Blackie Academic and Professional. Floros, J. D., Dock, L. L., Han, J. H. (1997). Active packaging technologies and applications. Food Cosmetics and Drug Packaging 20:10-17. Garde, J. A., Catala, R., Gavara, R., Hernandez, R. J. (2001). Characterising the migration of antioxidants into fatty food simulants. Food Additives and Contaminants 18:750-762. Hagelstein, A., Hoover, D., Paik, J., Kelley, M. (1995). Potential of antimicrobial nylon as a food package. Conference Proceedings, IFT Annual Meeting. Han, J. H. (2000). Antimicrobial food packaging. Food Technol. 54:56-65. Hansen, R., Rippl, C., Miidkiff, D., Neuwirth, J. (Jan. 11, 1988). Antimicrobial Absorbent Food Pad. U.S. Pat. No. 4,865,855. Hotchkiss, J. H. (1995). Safety considerations in active packaging. In: Rooney, M. L., ed. Active Food Packaging. London: Blackie Academic and Professional. Ikushima, K., Yashiki, I., Kuwabara, N., Hara, K., Hashimoto, H., Okura, I. (1994). Development of CD inclusion flavor essences, horseradish essences, menthol and ethanol for food additives. J. Appl. Glycosci. 41(2):197-200. Katan, L. L. (1996). Plastics. In: Migration from Food Contact Materials. London: Blackie Academic & Professional. Kim, H. J., Lee, S. C. (2002). Antimicrobial activity of silver ion against Salmonella Typhimurium, Staphylococcus Aureus and Vibrio Parahaemolyticus. Korean Soc. Food Sci. Nutr. 31(6):1163-1166. Kim, Y. M., An, D. S., Park, H. J., Park, J. M., Lee, D. S. (2002). Properties of nisin incorporated polymer coatings as antimicrobial packaging materials. Packaging Technol. Sci. 15:247-254. Kim, K. W., Thomas, R. L., Lee, C., Park, H. J. (2003). Antimicrobial activity of native chitosan, degraded chitosan and O-carboxymethylated chitosan. J. Food Protection 66:1495-1498. Labuza, T. P., Breene, W. M. (1989). Application of active packaging for improvement of shelf-life and nutritional quality of fresh and extended shelf-life foods. J. Food Processing and Preservation. 13:1-69. Ouattara, B., Simard, R. E., Piette, G., Begin, A., Holley, R. A. (2000a). Diffusion of acetic and propionic acids from chitosan-based antimicrobial packaging films. J. Food Sci. 65(5):768-773. Ouattara, B., Simard, R. E., Piette, G., Begin, A., Holley, R. A. (2000b). Inhibition of surface spoilage bacteria in processed meats by application of antimicrobial films prepared with chitosan. Int. J. Food Microbiol. 62:139-148. Quintavalla, S., Vicini, L. (2002). Antimicrobial food packaging in meat industry. Meat Sci. 62:373-380. Reineccius, T. A., Reineccius, G. A., Peppard, T. L. (2002). Encapsulation of flavors using cyclodextrins comparison of flavor retention in alpha, beta, and gamma types. J. Food Sci. 67(9):3271-3279. Sherrill, J., Michielsen, S., Stojiljkovic, I. (2003). Grafting of light-activated antimicrobial materials to nylon films. J. Polymer Sci. Part A: Polymer Chem. 41:41-47. Soares, N. F. F., Rutishauser, D. M., Melo, N., Cruz, R. S., Andrade, N. J. (2002). Inhibition of microbial growth in bread through active packaging. Packaging Technol. Sci. 15:129-132. Takayama, M., Sugimoto, H., Uchida, R., Yamauchi, R., Tanno, K. (1994). Antimicrobial activities of silver and copper ions. J. Antibacterial Antifungal Agents Japan 22(9):531-536. Tang, R., Du, Y., Fan, L. (2003). Dialdehyde starch-crosslinked chitosan films and their antimicrobial effects. J. Polymer Sci. 41:993-997. Vermeiren, L., Devlieghere, F., Beest, M. V., Kruijf, N. D., Debevere, J. (1999). Developments in the active packaging of foods. Trends Food Sci. Technol. 10:77-86. Vermeiren, L., Devlieghere, F., Debevere, J. (2002). Effectiveness of some recent antimicrobial packaging concepts. Food Additives and Contaminants. 19:163-171. Wirtanen, G., Aalto, M., Harkonen, P., Gilbert, P., Mattila-Sandholm, T. (2001). Efficacy testing of commercial disinfectants against foodborne pathogenic and spoilage microbes in biofilm-constructs. Eur. Food Res. Technol. 213(4/5): 409-414.

Although, antimicrobial polymers exist in the art, there is still a need for an improved antimicrobial polymer coating that may be easily and cheaply applied to a substrate to provide an article which has excellent antimicrobial properties and which retains its antimicrobial properties in a permanent and non-leachable fashion when in contact with cellular material for prolonged periods.

US patent application 20050271780 teaches a bactericidal polymer matrix being bound to an ion exchange material such as a quaternary ammonium salt for use in food preservation. This polymer matrix kills bacteria by virtue of incorporating therein of a bactericidal agent (e.g. the quaternary ammonium salt). The positive charge of the agent merely aids in electrostatic attraction between itself and the negatively charged cell walls. In addition, the above described application does not teach use of solid buffers having a buffering capacity throughout their entire body.

US patent application 20050249695 teaches immobilization of antimicrobial molecules such as quaternary ammonium or phosphonium salts (cationic, positively charged entities) covalently bound onto a solid surface to render the surface bactericidal. The polymers described herein are attached to a solid surface by virtue of amino groups attached thereto and as such the polymer is only capable of forming a monolayer on the solid surface.

US patent application 20050003163 teaches substrates having antimicrobial and/or antistatic properties. Such properties are imparted by applying a coating or film formed from a cationically-charged polymer composition.

The activity of the polymers as described in US patent applications 20050271780, 20050249695 and 20050003163 relies on the direct contact of the bactericidal materials with the cellular membrane. The level of toxicity is strongly dependent on the surface concentration of the bactericidal entities. This requirement presents a strong limitation since the exposed cationic materials can be saturated very fast in ion exchange reactions.

In addition, none of the above described US patent applications teach killing mammalian cells. Nor do they teach the in vivo use of polymers as cytotoxic agents against either eukaryotic or prokaryotic cell types. Furthermore, none of the above mentioned US patent applications teach configuration of the polymers to selectively kill certain cell types.

There thus remains a need for and it would be highly advantageous to have agents capable of cytotoxic action both against eukaryotic and prokaryotic cells.

### SUMMARY OF THE INVENTION

It is hence one object of the invention to disclose a biocidic packaging, especially a packaging for cosmetics and foodstuffs, comprising at least one insoluble proton sink or source (PSS). The packaging is provided useful for killing living target cells (LTCs), or otherwise disrupting vital intracellular processes and/or intercellular interactions of said LTC upon contact. The PSS comprising (i) proton source or sink providing a buffering capacity; and (ii) means providing proton conductivity and/or electrical potential; wherein said PSS is effectively disrupting the pH homeostasis and/or electrical balance within the confined volume of said LTC and/or disrupting vital intercellular interactions of said LTCs while efficiently preserving the pH of said LTCs' environment.

It is in the scope of the invention wherein the PSS is an insoluble hydrophobic, either anionic, cationic or zwitterionic charged polymer, useful for killing living target cells (LTCs), or otherwise disrupting vital intracellular processes and/or intercellular interactions of the LTC upon contact. It is additionally or alternatively in the scope of the invention, wherein the PSS is an insoluble hydrophilic, anionic, cationic or zwitterionic charged polymer, combined with water-immiscible polymers useful for killing living target cells (LTCs), or otherwise disrupting vital intracellular processes and/or intercellular interactions of the LTC upon contact. It is further in the scope of the invention, wherein the PSS is an insoluble hydrophilic, either anionic, cationic or zwitterionic charged polymer, combined with water-immiscible either anionic, cationic of zwitterionic charged polymer useful for killing living target cells (LTCs), or otherwise disrupting vital intracellular processes and/or intercellular interactions of the LTC upon contact.

It is also in the scope of the invention wherein the PSS is adapted in a non-limiting manner, to contact the living target cell either in a bulk or in a surface; e.g., at the outermost boundaries of an organism or inanimate object that are capable of being contacted by the PSS of the present invention; at the inner membranes and surfaces of microorganisms, animals and plants, capable of being contacted by the PSS by any of a number of transdermal delivery routes etc; at the bulk, either a bulk provisioned with stirring or nor etc.

It is further in the scope of the invention wherein either (i) a PSS or (ii) an article of manufacture comprising the PSS also comprises an effective measure of at least one additive

It is in the scope of the invention wherein the packaging is especially adapted to be provided as a packaging for cosmetics and foodstuffs, yet it is well in the scope of the invention wherein the packaging as hereinafter defined is utilizes for packaging other materials, e.g., any other compositions and products in solid, fluid or gas states.

It is another object of the invention to disclose biocidic packaging as defined in any of the above, wherein said proton conductivity is provided by water permeability and/or by wetting, especially wherein said wetting is provided by hydrophilic additives.

It is another object of the invention to disclose biocidic packaging as defined in any of the above, wherein said proton conductivity or wetting is provided by inherently proton conductive materials (IPCMs) and/or inherently hydrophilic polymers (IHPs), selected from a group consisting of sulfonated tetrafluortheylene copolymers; sulfonated materials selected from a group consisting of silica, polythion-ether sulfone (SPTES), styrene-ethylene-butylene-styrene (S-SEBS), polyether-ether-ketone (PEEK), poly(arylene-ether-sulfone) (PSU), Polyvinylidene Fluoride (PVDF)-grafted styrene, polybenzimidazole (PBI) and polyphosphazene; proton-exchange membrane made by casting a polystyrene sulfonate (PSSnate) solution with suspended micron-sized particles of cross-linked PSSnate ion exchange resin; commercially available Nafion™ and derivatives thereof.

It is another object of the invention to disclose biocidic packaging as defined in any of the above, wherein it is constructed as a conjugate, comprising two or more, either two-dimensional (2D) or three-dimensional (3D) PSSs, each of which of the PSSs consisting of materials containing highly dissociating cationic and/or anionic groups (HDCAs) spatially organized in a manner which efficiently minimizes the change of the pH of the LTC's environment. Each of the HDCAs is optionally spatially organized in specific either 2D, topologically folded 2D surfaces, or 3D manner efficiently which minimizes the change of the pH of the LTC's environment; further optionally, at least a portion of the spatially organized HDCAs are either 2D or 3D positioned in a manner selected from a group consisting of (i) interlacing; (ii) overlapping; (iii) conjugating; (iv) either homogeneously or heterogeneously mixing; and (iv) tiling the same.

It is acknowledged in this respect to underline that the term HDCAs refers, according to one specific embodiment of the invention, and in a non-limiting manner, to ion-exchangers, e.g., water immiscible ionic hydrophobic materials.

It is another object of the invention to disclose biocidic packaging as defined in any of the above, wherein said PSS is effectively disrupting the pH homeostasis within a confined volume while efficiently preserving the entirety of said LTC's environment, especially a cosmetic article or a foodstuff; and further wherein said environment's entirety is characterized by parameters selected from a group consisting of said environment functionality, chemistry; soluble's concentration, possibly other then proton or hydroxyl concentration; biological related parameters; ecological related parameters; physical parameters, especially particles size distribution, rheology and consistency; safety parameters, especially toxicity, otherwise LD₅₀ or ICT₅₀ affecting parameters; olphactory or organoleptic parameters (e.g., color, taste, smell, texture, conceptual appearance etc); or any combination of the same.

It is another object of the invention to disclose a biocidic packaging as defined in any of the above, provided useful for disrupting vital intracellular processes and/or intercellular interactions of said LTC, while both (i) effectively preserving the pH of said LTC's environment, especially a cosmetic article of a foodstuff, and (ii) minimally affecting the entirety of the LTC's environment such that a leaching from said PSS of either ionized or neutral atoms, molecules or particles to the LTC's environment is minimized.

It is well in the scope of the invention wherein the aforesaid leaching minimized such that the concentration of leached ionized or neutral atoms is less than 1 ppm. Alternatively, the aforesaid leaching is minimized such that the concentration of leached ionized or neutral atoms is less than less than 50 ppb. Alternatively, the aforesaid leaching is minimized such that the concentration of leached ionized or neutral atoms is less than less than 50 ppb and more than 10 ppb. Alternatively, the aforesaid leaching is minimized such that the concentration of leached ionized or neutral atoms is less than less than 10 but more than 0.5 ppb. Alternatively, the aforesaid leaching is minimized such that the concentration of leached ionized or neutral atoms is less than less than 0.5 ppb.

It is another object of the invention to disclose a biocidic packaging as defined in any of the above, provided useful for disrupting vital intracellular processes and/or intercellular interactions of said LTC, while less disrupting pH homeostasis and/or electrical balance within at least one second confined volume (e.g., non-target cells or viruses, NTC).

It is another object of the invention to disclose a biocidic packaging as defined in any of the above, wherein said differentiation between said LTC and NTC is obtained by one or more of the following means (i) differential ion capacity; (ii) differential pH values; and, (iii) optimizing PSS to target cell size ratio; (iv) providing a differential spatial, either 2D, topologically 2D folded surfaces, or 3D configuration of the PSS; (v) providing a critical number of PSS' particles (or applicable surface) with a defined capacity per a given volume; and (vi) providing size exclusion means.

It is another object of the invention to disclose biocidic packaging for cosmetics and foodstuffs, comprising at least one insoluble non-leaching PSS as defined in any of the above; said PSS, located on the internal and/or external surface of said packaging, is provided useful, upon contact, for disrupting pH homeostasis and/or electrical balance within at least a portion of an LTC while effectively preserving pH & functionality of said surface.

It is another object of the invention to disclose a biocidic packaging as defined in any of the above, having at least one external proton-permeable surface with a given functionality (e.g., electrical current conductivity, affinity, selectivity etc), said surface is at least partially composed of, or topically and/or underneath layered with a PSS, such that disruption of vital intracellular processes and/or intercellular interactions of said LTC is provided, while said LTC's environment's pH & said functionality is effectively preserved.

It is another object of the invention to disclose a biocidic packaging as defined in any of the above, comprising a surface with a given functionality, and one or more external proton-permeable layers, each of which of said layers is disposed on at least a portion of said surface; wherein said layer is at least partially composed of or layered with a PSS such that vital intracellular processes and/or intercellular interactions of said LTC are disrupted, while said LTC's environment's pH & said functionality is effectively preserved.

It is another object of the invention to disclose a biocidic packaging as defined in any of the above, comprising (i) at least one PSS; and (ii) one or more preventive barriers, providing said PSS with a sustained long activity; preferably wherein at least one barrier is a polymeric preventive barrier adapted to avoid heavy ion diffusion; further preferably wherein said polymer is an ionomeric barrier, and particularly a commercially available Nafion™.

It is acknowledged in this respect that the presence or incorporation of barriers that can selectively allow transport of protons and hydroxyls but not of other competing ions to and/or from the solid ion exchangers (SIEx) surface eliminates or substantially reduces the ion-exchange saturation by counter-ions, resulting in sustained and long acting cell killing activity of the materials and compositions of the current invention.

It is in the scope of the invention, wherein the proton and/or hydroxyl-exchange between the cell and strong acids and/or strong basic materials and compositions may lead to disruption of the cell pH-homeostasis and consequently to cell death. The proton conductivity property, the volume buffer capacity and the bulk activity are pivotal and crucial to the present invention.

It is further in the scope of the invention, wherein the pH derived biocidic activity can be modulated by impregnation and coating of acidic and basic ion exchange materials with polymeric and/or ionomeric barrier materials.

It is another object of the invention to disclose a biocidic packaging as defined in any of the above, wherein the packaging is adapted to avoid development of LTC's resistance and selection over resistant mutations.

It is another object of the invention to disclose a biocidic packaging as defined in any of the above, wherein the packaging is designed as a continuous barrier said barrier is selected from a group consisting of either 2D or 3D membranes, filters, meshes, nets, sheet-like members or a combination thereof.

It is another object of the invention to disclose a biocidic packaging as defined in any of the above, wherein the packaging is as an insert, comprising at least one. PSS, said insert is provided with dimensions adapted to ensure either (i) reversibly mounting or (ii) permanent accommodation of said insert within a predetermined article of manufacture.

It is in the scope of the invention, wherein the insert is constructed as a sheet-like member (e.g., dip like member etc) or as a particulated (bulky) matter, such as a porosive powder. The insert may be a stand-alone product, or it may have a secondary functionality, such as a twisted cork of a bottle, a removable flexible sealing of a food container. The insert is selected by its surface area, or by its effective volume.

It is another object of the invention to disclose a biocidic packaging as defined in any of the above, wherein the packaging is characterized by at least one of the following (i) regeneratable proton source or sink; (ii) regeneratable buffering capacity; and (iii) regeneratable proton, conductivity.

It is another object of the invention to disclose a method for killing living target cells (LTCs), or otherwise disrupting vital intracellular processes and/or intercellular interactions of said LTC being in a packaging, especially cosmetic or foodstuffs' packaging; said method comprising steps of: providing said packaging with at least one PSS having (i) proton source or sink providing a buffering capacity; and (ii) means providing proton conductivity and/or electrical potential; contacting said LTCs with said PSS; and, by means of said PSS, effectively disrupting the pH homeostasis and/or electrical balance within said LTC while efficiently preserving the pH of said LTC's environment.

It is another object of the method as defined in any of the above, wherein said step (a) further comprising a step of providing said PSS with water permeability and/or wetting characteristics, in particular wherein said proton conductivity and wetting is at least partially obtained by providing said PSS with hydrophilic additives.

It is another object of the invention is to disclose a method as defined in any of the above, wherein the method further comprising a step of providing the PSS with inherently proton conductive materials (IPCMs) and/or inherently hydrophilic polymers (IHPs), especially by selecting said IPCMs and/or IHPs from a group consisting of sulfonated tetrafluoroethethylene copolymers; commercially available Nafion™ and derivatives thereof.

It is another object of the invention is to disclose a method as defined in any of the above, wherein the method further comprising steps of providing the packaging with two or more, either two-dimensional (2D), topologically folded 2D surfaces or three-dimensional (3D) PSSs, each of which of said PSSs consisting of materials containing highly dissociating cationic and/or anionic groups (HDCAs); and, spatially organizing said HDCAs in a manner which minimizes the change of the pH of the LTC's environment, especially a cosmetic article of a foodstuff.

It is another object of the invention is to disclose a method as defined in any of the above, wherein the method further comprising a step of spatially organizing each of said HDCAs in a specific, either 2D or 3D manner, such that the change of the pH of the LTC's environment is minimized.

It is another object of the invention is to disclose a method as defined in any of the above, wherein said step of organizing is provided by a manner selected for a group consisting of (i) interlacing said HDCAs; (ii) overlapping said HDCAs; (iii) conjugating said HDCAs; and (iv) either homogeneously or heterogeneously mixing said HDCAs; and (v) tiling of the same.

It is another object of the invention is to disclose a method as defined in any of the above, wherein the method further comprising a step of disrupting pH homeostasis and/or electrical potential within at least a portion of an LTC by a PSS, while both (i) effectively preserving the pH of said LTC's environment, especially a cosmetic article of a foodstuff; and (ii) minimally affecting the entirety of said LTC's environment; said method is especially provided by minimizing the leaching of either ionized or electrically neutral atoms, molecules or particles (AMP) from the PSS to said LTC's environment.

It is another object of the invention is to disclose a method as defined in any of the above, wherein the method further comprising steps of preferentially disrupting pH homeostasis and/or electrical balance within at least one first confined volume (e.g., target living cells or viruses, LTC), while less disrupting pH homeostasis within at least one second confined volume (e.g., non-target cells or viruses, NTC).

It is another object of the invention is to disclose a method as defined in any of the above, wherein the method wherein a differentiation between said LTC and NTC is obtained by one or more of the following steps: (i) providing differential ion capacity; (ii) providing differential pH value; (iii) optimizing the PSS to LTC size ratio; and, (iv) designing a differential spatial configuration of said PSS boundaries on top of the PSS bulk; and (v) providing a critical number of PSS' particles (or applicable surface) with a defined capacity per a given volume; and (vi) providing size exclusion means, e.g., mesh, grids etc.

It is another object of the invention is to disclose a method for the production of a biocidic packaging for cosmetics and/or foodstuffs, comprising steps of providing a packaging as defined in as defined above; locating the PSS on top or underneath the surface of said packaging; and upon contacting said PSS with a LTC, disrupting the pH homeostasis and/or electrical balance within at least a portion of said LTC while effectively preserving pH & functionality of said surface.

It is another object of the invention is to disclose a method as defined in any of the above, wherein the method further comprising steps of providing the packaging with at least one external proton-permeable surface with a given functionality; and, providing at least a portion of said surface with at least one PSS, and/or layering at least one PSS on top of underneath said surface; hence killing LTCs or otherwise disrupting vital intracellular processes and/or intercellular interactions of said LTC, while effectively preserving said LTC's environment's pH & functionality.

It is another object of the invention is to disclose a method as defined in any of the above, wherein the method further comprising steps of providing the packaging with at least one external proton-permeable providing a surface with a given functionality; disposing one or more external proton-permeable layers topically and/or underneath at least a portion of said surface; said one or more layers are at least partially composed of or layered with at least one PSS; and, killing LTCs, or otherwise disrupting vital intracellular processes and/or intercellular interactions of said LTC, while effectively preserving said LTC's environment's pH & functionality.

It is another object of the invention is to disclose the method as defined in any of the above, wherein the method comprising steps of providing the packaging with at least one PSS; and, providing said PSS with at least one preventive barrier such that a sustained long acting is obtained.

It is another object of the invention is to disclose a method as defined in any of the above, wherein said step of providing said barrier is obtained by utilizing a polymeric preventive barrier adapted to avoid heavy ion diffusion; preferably by providing said polymer as an ionomeric barrier, and particularly by utilizing a commercially available Nafion™ product.

It is another object of the invention is to disclose a method for inducing apoptosis in at least a portion of LTCs population in a packaging, especially a packaging of cosmetics and foodstuffs; said method comprising steps of obtaining at least one packaging as defined above, contacting the PSS with an LTC; and, effectively disrupting the pH homeostasis and/or electrical balance within said LTC such that said LTC's apoptosis is obtained, while efficiently preserving the pH of said LTC's environment and patient's safety.

It is hence in the scope of the invention wherein one or more of the following materials are provided: encapsulated strong acidic and strong basic buffers in solid or semi-solid envelopes, solid ion-exchangers (SIEx), ionomers, coated-SIEx, high-cross-linked small-pores SIEx, Filled-pores SIEx, matrix-embedded SIEx, ionomeric particles embedded in matrices, mixture of anionic (acidic) and cationic (basic) SIEx etc.

It is another object of the invention to disclose the PSS as defined in any of the above, wherein the PSS are naturally occurring organic acids compositions containing a variety of carbocsylic and/or sulfonic acid groups of the family, abietic acid (C₂₀H₃₀O₂) such as colophony/rosin, pine resin and alike, acidic and basic terpenes.

It is another object of the invention is to disclose a method for avoiding development of LTC's resistance and selecting over resistant mutations, said method comprising steps of: obtaining at least one packaging as defined above; contacting the PSS with an LTC; and, effectively disrupting the pH homeostasis and/or electrical balance within said LTC such that development of LTC's resistance and selecting over resistant mutations is avoided, while efficiently preserving the pH of said LTC's environment, especially a cosmetic article or a foodstuff.

It is another object of the invention is to disclose a method of regenerating the biocidic properties of a packaging as defined above; comprising at least one step selected from a group consisting of (i) regenerating said PSS; (ii) regenerating its buffering capacity; and (iii) regenerating its proton conductivity.

### BRIEF DESCRIPTION OF THE DRAWINGS

In order to understand the invention and to see how it may be implemented in practice, a plurality of preferred embodiments will now be described, by way of non-limiting example only, with reference to the accompanying drawing, in which

FIG. 1 is presenting bacterial count of E. coli in Nafion™ coated vs. uncoated vials;

FIG. 2 is showing the comparison of bacterial deposit in uncoated (left) vs. coated vial (right);

FIG. 3 is illustrating the bacterial growth inhibition (S. aureus) in Dormin™ solution;

FIG. 4 is showing the bacterial growth inhibition (E. coli) in Dormin™ solution;

FIG. 5 is showing a bacterial development in cosmetic cream in Nafion™ coated dishes;

FIG. 6 is presenting the bacterial development in cosmetic cream in Nafion™ coated dishes;

FIG. 7 is illustrating the biofilm count on control and coated glass slides. The antifouling property of the G5 composition was evaluated using standard bacteriological test; Bacteriological samples were obtained from the glass using a swab seeded, and counted;

FIG. 8 is presenting the media bacterial load. Media bacterial load was measured after 3, 11 and 13 days of incubation; the media was sampled, seeded, incubated and counted;

FIG. 9 is displaying a photograph of the media turbidity—representative growing media picture after 3 days of incubation;

FIG. 10 is illustrating the effect of BioActivity™ coating of glass vessels on S. caseolyticus-inoculated UHT milk; and,

FIG. 11 is displaying the pH dynamics of fruit juice stored in BioActivity™ laminated containers and in control container for 14 days at room temperature.

### DESCRIPTION OF THE PREFERRED EMBODIMENTS

The following specification taken in conjunction with the drawings sets forth the preferred embodiments of the present invention. The embodiments of the invention disclosed herein are the best modes contemplated by the inventors for carrying out their invention in a commercial environment, although it should be understood that various modifications can be accomplished within the parameters of the present invention.

The term ‘contact’ refers hereinafter to any direct or indirect contact of a PSS with a confined volume (living target cell or virus—LTC), wherein said PSS and LTC are located adjacently, e.g., wherein the PSS approaches either the internal or external portions of the LTC; further wherein said PSS and said LTC are within a proximity which enables (i) an effective disruption of the pH homeostasis and/or electrical balance, or (ii) otherwise disrupting vital intracellular processes and/or intercellular interactions of said LTC.

The terms ‘effectively’ and ‘effectively’ refer hereinafter to an effectiveness of over 10%, additionally or alternatively, the term refers to an effectiveness of over 50%; additionally or alternatively, the term refers to an effectiveness of over 80%. It is in the scope of the invention, wherein for purposes of killing LTCs, the term refers to killing of more than 50% of the LTC population in a predetermined time, e.g., 10 min.

The term ‘additives’ refers hereinafter to one or more members of a group consisting of biocides e.g., organic biocides such as tea tree oil, rosin, abietic acid, terpens, rosemary oil etc, and inorganic biocides, such as zinc oxides, copper and mercury, silver salts etc, markers, biomarkers, dyes, pigments, radio-labeled materials, glues, adhesives, lubricants, medicaments, sustained release drugs, nutrients, peptides, amino acids, polysaccharides, enzymes, hormones, chelators, multivalent ions, emulsifying or de-emulsifying agents, binders, fillers, thickfiers, factors, co-factors, enzymatic-inhibitors, organoleptic agents, carrying means, such as liposomes, multilayered vesicles or other vesicles, magnetic or paramagnetic materials, ferromagnetic and non-ferromagnetic materials, biocompatibility-enhancing materials and/or biodegradating materials, such as polylactic acids and polyglutamine acids, anticorrosive pigments, anti-fouling pigments, UV absorbers, UV enhancers, blood coagulators, inhibitors of blood coagulation, e.g., heparin and the like, or any combination thereof.

The term ‘particulate matter’ refers hereinafter to one or more members of a group consisting of nano-powders, micrometer-scale powders, fine powders, free-flowing powders, dusts, aggregates, particles having an average diameter ranging from about 1 nm to about 1000 nm, or from about 1 mm to about 25 mm.

The term about’ refers hereinafter to ±20% of the defined measure.

The term ‘cosmetics’ refers hereinafter in a non-limiting manner to eye shadows, blushers, bronzers, foundations and other products, presented in a powder or creamy powder or creamy final form, which are applied to parts of the human body for purposes of enhancing appearance, lipsticks or other hot pour liquid products. Cosmetics can be either liquid or powder. The term also refers to make-up, foundation, and skin care products. The term “make-up” refers to products that leave color on the face, including foundation, blacks and browns, i.e., mascara, concealers, eye liners, brow colors, eye shadows, blushers, lip colors, powders, solid emulsion compact, and so forth. “Skin care products” are those used to treat or care for, or somehow moisturize, improve, or clean the skin. Products contemplated by the phrase “skin care products” include, but are not limited to, adhesives, bandages, toothpaste; anhydrous occlusive moisturizers, antiperspirants, deodorants, personal cleansing products, powder laundry detergent, fabric softener towels, occlusive drug delivery patches, nail polish, powders, tissues, wipes, hair conditioners-anhydrous, shaving creams and the like. The term “foundation” refers to liquid, creme, mousse, pancake, compact, concealer or like product created or reintroduced by cosmetic companies to even out the overall coloring of the skin.

The term ‘foodstuffs’ refers hereinafter in a non-limiting manner to foodstuffs which have usually only been subjected to one processing step, often by the actual producer, before delivery to the consumer; e.g., meat such as meat of veal, roast beef, filet steak, entrecote; pork meat, minced meat, lambs meat, wild animal, chicken meat, and further including various prepared meat dishes in the form of stews and casseroles, liver and blood products, sauces, seafood and fish, and egg products. The term also refers to “Secondary foodstuff” i.e., foodstuff which has been further processed by a manufacturer en route from producer to consumer, such as vegetarian steaks, gratinated vegs, oven made lasagne, fish and ham with potatoes, meat pasta dishes, soups, hamburgers, pizzas, sausage products, pastries and bakery products, bread, milk product including cream, ice cream and cheese, hummus, tehina etc. The term also include any products: raw, prepared or processed, which are intended for human consumption in particular by eating or drinking and which may contain nutrients or stimulants in the form of minerals, carbohydrates (including sugars), proteins and/or fats. The term also refers to “functional foodstuffs or food compositions”. The term also used for unmodified food form. The term also refers to all bereaves, drinks, water-based solutions, water-immiscible solutions, extracts, and also to pure drinking water. The term shall be understood to mean any a liquid or solid a foodstuff.

The present invention relates to materials, compositions and methods for prevention of bacterial development in cosmetics by manufacturing packaging and closure mechanisms capable of inhibition of bacterial proliferation and biofilm formation. The antibacterial activity is based on preferential proton and/or hydroxyl-exchange between the cell and strong acids and/or strong basic materials and compositions. The materials and compositions of the present invention exert their antimicrobial and anti-biofilm effect via a titration-like process in which the said cell (bacteria, yeast, fungi etc.) is coming into contact with strong acids and/or strong basic buffers and the like: encapsulated strong acidic and strong basic buffers in solid or semi-solid envelopes, solid ion-exchangers (SIEx), ionomers, coated-SIEx, high-cross-linked small-pores SIEx, Filled-pores SIEx, matrix-embedded SIEx, Ionomeric particles embedded in matrices, mixture of anionic (acidic) and cationic (basic) SIEx etc. This process leads to disruption of the cell pH-homeostasis and consequently to cell death. The proton conductivity property, the volume buffer capacity and the bulk activity are pivotal and crucial to the present invention. The presence or incorporation of barriers that can selectively allow transport of protons and hydroxyls but not of other competing ions to and/or from the SIEx surface eliminates or substantially reduces the ion-exchange saturation by counter-ions, resulting in sustained and long acting cell killing activity of the materials and compositions of the current invention.

The materials and compositions of the current invention include but not limited to all materials and compositions disclosed in PCT application No. PCT/IL2006/001263.

The above mentioned materials and compositions of PCT/IL2006/001262 modified in such a way that these said compositions are ion-selective by, for example: coating them with a selective coating, or ion-selective membrane; coating or embedding in high-cross-linked size excluding polymers etc; Strong acidic and strong basic buffers encapsulated in solid or semi-solid envelopes; SIEx particles—coated and non-coated, alone or in a mixture, embedded in matrices so as to create a pH-modulated polymer; SIEx particles—coated and non-coated, embedded in porous ceramic or glass water permeable matrices; Polymers which are alternately tiled with areas of high and low pH to create a mosaic-like polymer with an extended cell-killing spectrum.

In addition to ionomers disclosed in the above mentioned PCT No. PCT/IL2006/001263, other ionomers can be used in the current invention as cell-killing materials and compositions. These may include, but certainly not limited to, for example: sulfonated silica, sulfonated polythion-ether sulfone (SPTES), sulfonated styrene-ethylene-butylene-styrene (S-SEBS), polyether-ether-ketone (PEEK), poly(arylene-ether-sulfone) (PSU), Polyvinylidene Fluoride (PVDF)-grafted styrene, polybenzimidazole (PBI) and polyphosphazene, proton-exchange membrane made by casting a polystyrene sulfonate (PSS) solution with suspended micron-sized particles of cross-linked PSS ion exchange resin.

All of the above mentioned materials and compositions of the current invention can be cast, molded or extruded and be used as particles in suspension, spray, as membranes, coated films, fibers or hollow fibers, paper, particles linked to or absorbed on fibers or hollow fibers, incorporated in filters or tubes and pipes etc.

It is in the scope of the invention, wherein biocidic packaging for cosmetics and foodstuffs comprises insoluble PSS in the form of a polymer, ceramic, gel, resin or metal oxide is disclosed. The PSS is carrying strongly acidic or strongly basic functional groups (or both) adjusted to a pH of about <4.5 or about >8.0. It is in the scope of the invention, wherein the insoluble PSS is a solid buffer.

It is also in the scope of the invention wherein material's composition is provided such that the groups are accessible to water whether they are on the surface or in the interior of the PSS. Contacting a living cell (e.g., bacteria, fungi, animal or plant cell) with the PSS kills the cell in a time period and with an effectiveness depending on the pH of the PSS, the mass of PSS contacting the cell, the specific functional group(s) carried by the PSS, and the cell type. The cell is killed by a titration process where the PSS causes a pH change within the cell. The cell is often effectively killed before membrane disruption or cell lysis occurs. The PSS kills cells without directly contacting the cells if contact is made through a coating or membrane which is permeable to water, H+ and OH− ions, but not other ions or molecules. Such a coating also serves to prevent changing the pH of the PSS or of the solution surrounding the target cell by diffusion of counterions to the PS S's functional groups. It is acknowledged in this respect that prior art discloses cell killing by strongly cationic (basic) molecules or polymers where killing probably occurs by membrane disruption and requires contact with the strongly cationic material or insertion of at least part of the material into the outer cell membrane.

It is also in the scope of the invention wherein an insoluble polymer, ceramic, gel, resin or metal oxide carrying strongly acid (e.g. sulfonic acid or phosphoric acid) or strongly basic (e.g. quaternary or tertiary amines) functional groups (or both) of a pH of about <4.5 or about >8.0 is disclosed. The functional groups throughout the PSS are accessible to water, with a volumetric buffering capacity of about 20 to about 100 mM H⁺/l/pH unit, which gives a neutral pH when placed in unbuffered water (e.g., about 5<pH>about 7.5) but which kills living cells upon contact.

It is also in the scope of the invention wherein the insoluble polymer, ceramic, gel, resin or metal oxide as defined above is coated with a barrier layer permeable to water, H⁺ and OH⁻ ions, but not to larger ions or molecules, which kills living cells upon contact with the barrier layer.

It is also in the scope of the invention wherein the insoluble polymer, ceramic, gel, resin or metal oxide as defined above is provided useful for killing living cells by inducing a pH change in the cells upon contact.

It is also in the scope of the invention wherein the insoluble polymer, ceramic, gel, resin or metal oxide as defined above is provided useful for killing living cells without necessarily inserting any of its structure into or binding to the cell membrane.

It is also in the scope of the invention wherein the insoluble polymer, ceramic, gel, resin or metal oxide as defined above is provided useful for killing living cells without necessarily prior disruption of the cell membrane and lysis.

It is also in the scope of the invention wherein the insoluble polymer, ceramic, gel, resin or metal oxide as defined above is provided useful for causing a change of about <0.2 pH units of a physiological solution or body fluid surrounding a living cell while killing the living cell upon contact.

It is also in the scope of the invention wherein the insoluble polymer, ceramic, gel, resin or metal oxide as defined above is provided in the form of shapes, a coating, a film, sheets, beads, particles, microparticles or nanoparticles, fibers, threads, powders and a suspension of these particles.

It is also anticipated that the above described materials and compositions would be incorporated into or be part of the packaging cup, lid stopper or seal; inserted into the package by any sort of inserts such as membranes, wraps, separating sheets and foils, rods, picks, mesh, spheres, beads, buoys, floats, rings etc.

The above materials have proven high antibacterial activity when used in food packaging trials for foodstuff like milk, fruit juice, meat etc.

The current invention is based on the modification of the internal surfaces of the cosmetic containers, tubes, jars, bottles etc. with a thin layer of the materials of invention to prevent bacterial development on the internal container surface.

Those coatings can be produced by methods known in industry like spin coating, internal spray processing, Thermoplastic spraying, Evaporative deposition, coating with a varnish or thin layer resin etc and can be deposited on surfaces of polymers, glass, paper or any other material.

In all these coatings the active antibacterial materials will be incorporated in a polymer matrix suitable for attachment on the container material.

### Example 1

### Comparison of Bacterial Development (E. coli) in TSB in Vials Coated with Nafion™ vs. Uncoated Vials

### Materials and Methods

15 ml vials were coated with commercial solution of Nafion™ (commercially available product of Du Pont) and left to dry. This generated a thin-layer (˜50 microns) of polymerized Nafion™ on the internal surface of the vial.

Coated and uncoated vials were filled with 10 ml of TSB and inoculated with E. coli (3×10⁶ cfu/ml). Vials were than incubated in a stationary incubation at 30° C. Bacterial count (cfu/ml) was measured at time zero and 3 hours and 3 days after inoculation by sampling the and dispersing bacterial broth on TSA plates and counting 24 hours later incubation at 30° C.

### Results

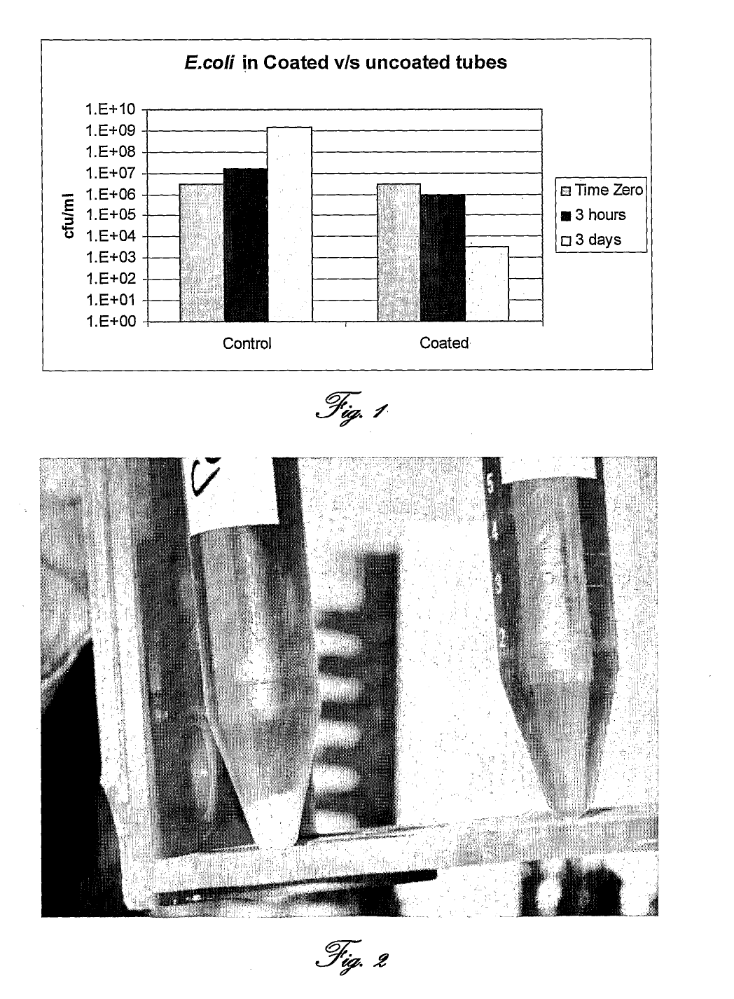

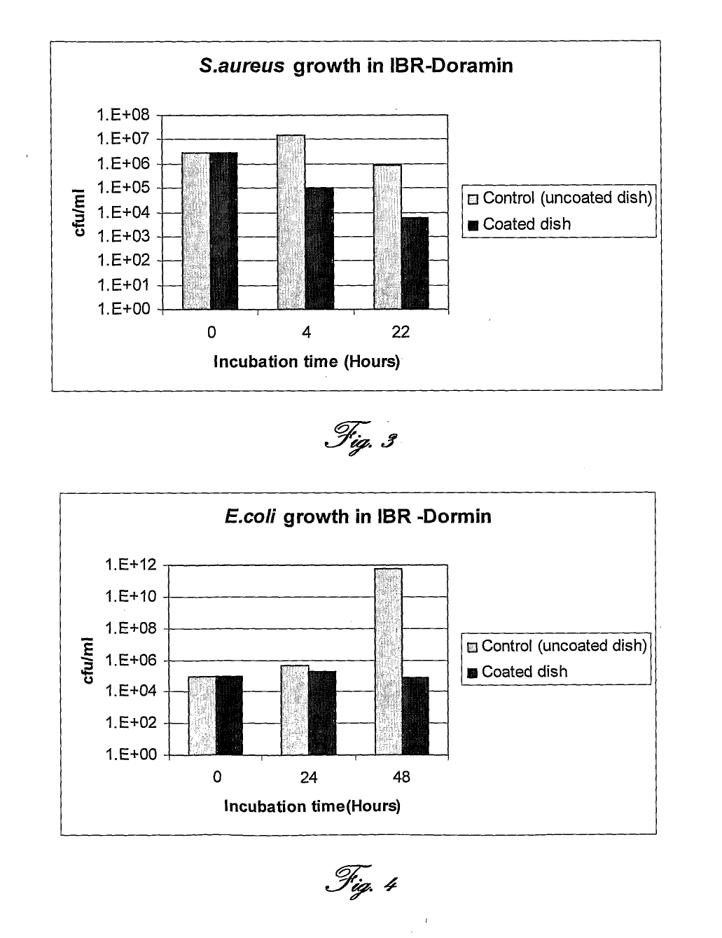

Reference is now made to FIG. 1 presenting bacterial count of E. coli in Nafion™ coated vs. uncoated vials; and to FIG. 2 showing the comparison of bacterial deposit in uncoated (left) vs. coated vial (right).

In the uncoated control, bacterial counts increased starting after 3 hours of incubation and reaching a level of 10⁹ cfu/ml after 3 days (See FIG. 1). On the other hand, Nafion™ coated vials showed strong inhibition and antibacterial activity resulting in decline in bacterial counts to a level of ˜5×10³ cfu/ml after 3 days.

FIG. 2 shows the lack of bacterial deposition in the Nafion™-coated vial as compared to the clearly visible deposited bacteria in the uncoated tube.

### Example 2

### Bacterial Development in Dormin™ in Coated vs. Uncoated Vials

Dormins are natural extracts from plants and plant organs in their dormant stage which are able to slow down cell proliferation, maintain younger healthier skin and provide the means for better skin protection. Dormins are being utilized by many cosmetic Companies as active ingredients in cosmetic creams and lotions. Dormins are susceptible to bacterial and fungal contamination.

### Materials and Methods

In the experiment 100 microliters of Staphylococcus aureus culture at a concentration of 5.8×10⁷ cfu/ml were added to 2 ml of preservatives-free Dormin™ solution (obtained from IBR, Rehovot, Israel). S. aureus inoculated Dormin™ solution was deposited into a culture dish coated with a 50 micrometer-thick layer of Nafion™. Bacterial proliferation was monitored after 4 and 22 hours of incubation at 30° C. by plating samples on TSA plates and incubation for 24 hrs at 30° C.

### Results

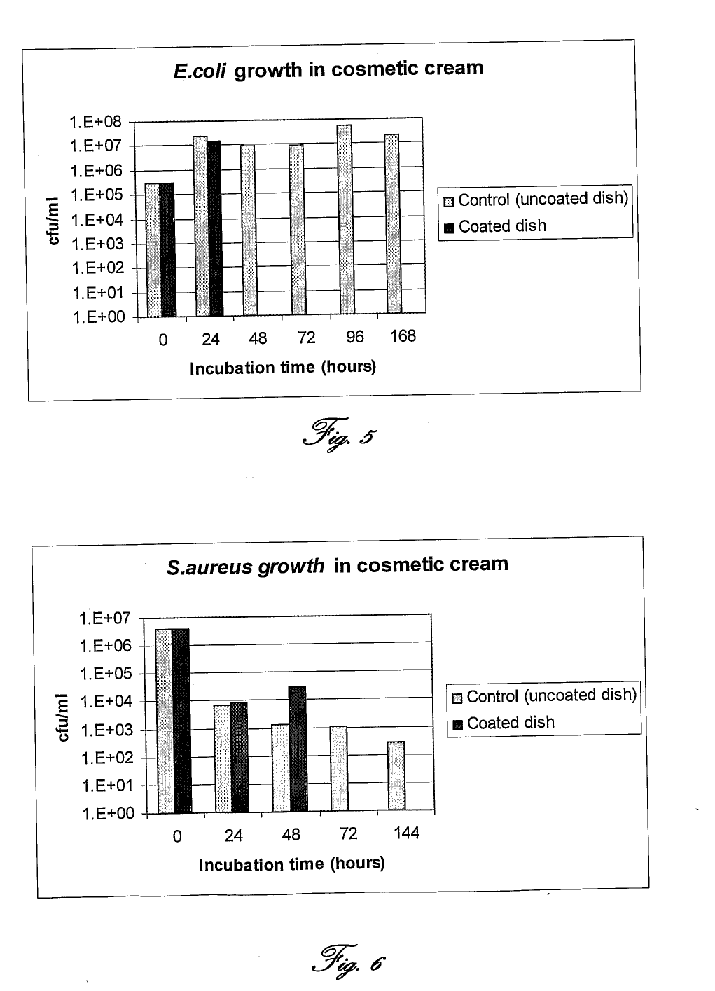

Reference is now made to FIG. 3 illustrating the bacterial growth inhibition (S. aureus) in Dormin™ solution; and to FIG. 4, showing the bacterial growth inhibition (E. coli) in Dormin™ solution

The results show a strong inhibition of bacterial development in the Dormin™ solution incubated at the presence of 50 micrometer-thick layer of Nafion™ as oppose to the untreated control (FIG. 3)

A similar experiment was performed with E. coli showing again the strong inhibition effect of the active coating as seen in FIG. 4.

### Example 3

### Bacterial Inhibition in, Preservatives-Free, Commercial Cosmetic Cream

### Materials and Methods

Samples of commercially available cosmetic cream, free of preservatives, were obtained from IBR Ltd., Rehovot, Israel. Starter cultures of E. coli and S. aureus were grown on TSB for 4 hrs at 30° C. and mixed with the cosmetic cream at 1:1 ratio (8 ml of each culture were mixed with 8 grams of cosmetic cream) and deposited in Nafion™ coated dishes. Bacterial development was monitored as described above at time intervals of 0, 24, 48, 72, 96, 144 and 168 hrs of incubation at 30° C.

### Results

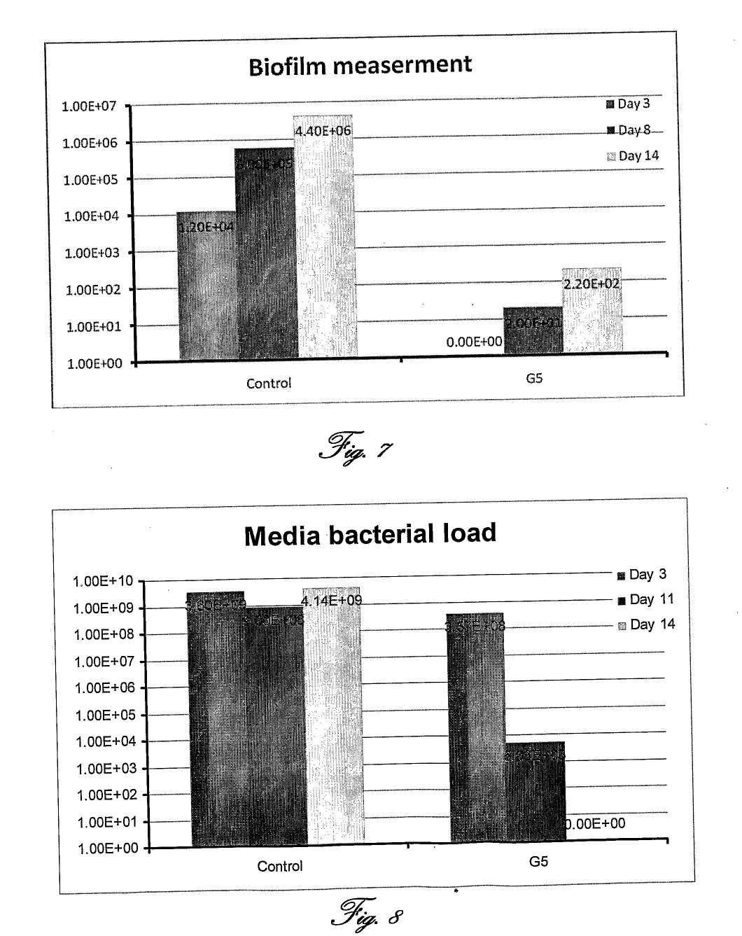

Reference is now made to FIG. 5, showing a bacterial development in cosmetic cream in Nafion™ coated dishes; and to FIG. 6, presenting the bacterial development in cosmetic cream in Nafion™ coated dishes.

FIGS. 5 and 6 shows strong bacterial growth inhibition in the cosmetic cream kept in the Nafion™ coated dishes as compared to the uncoated. Practically no E. coli and S. aureus could be recovered from the cream kept in the Nafion™ coated dishes after 48 hrs and 72 hrs, respectively.

### Example 4

### Biofilm Prevention in Liquids Using Antibacterial Inserts

### Materials and Methods

The antifouling properties of compositions G5 was evaluated using a closed-aerobic system. polystyrene (PS) slides were coated with G5 [Sulfonated silica 10%, Potassium sulfate 5%, Potassium laurate 10%, Mineral oil 65%, paraffin (white)] and incubated vertically in 50 ml perforated tubes (30° C., 50 rpm) with E. coli (10⁶ c.f.u/ml TSB). In order to maintain the nutrient level in the media, every 3 days 10 ml media was replaced with fresh media. During 14 days of incubation, the antifouling property of the G5-composition was evaluated using standard bacteriological test. Bacteriological samples were obtained from the glass. Slides were taken out of the tube, washed in dw water, and dried (1 h, RT) prior to sampling. Using a swab, 1 cm samples were obtained, the cotton of the swab was soaked in PBS 500 μl, shaken vigorously, and diluted into decimal dilutions (bacterial samples 100 μl) seeded on TSA petri dish (Hy labs, Israel), incubated (30° C., 48 h) and counted. In order to study the effect of the coated glass on bacterial load in the surrounding media, the media was sampled as well (primary bacterial samples 100 μl) diluted using serial decimal dilutions with PBS, seeded on petri dishes (TSA Petri dish), incubated (30° C., 24 h) and counted.

### Results

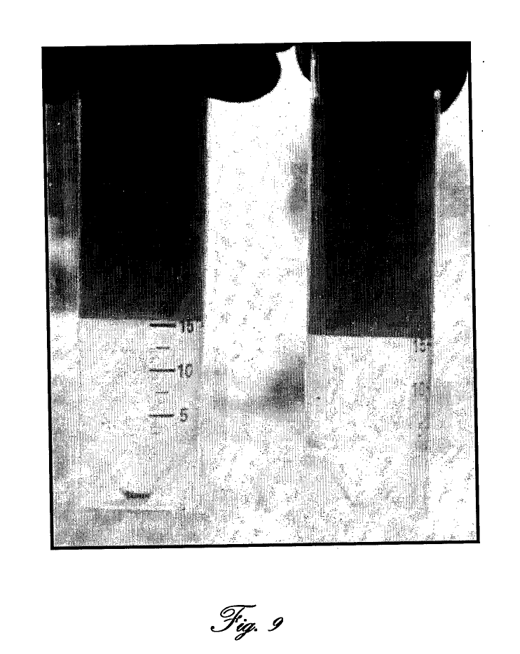

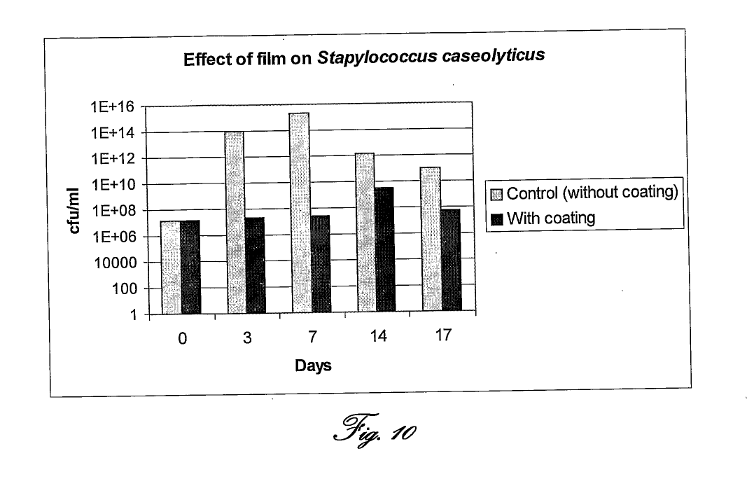

Reference is now made to FIG. 7, illustrating the biofilm count on control and coated glass slides. The antifouling property of the G5 composition was evaluated using standard bacteriological test. Bacteriological samples were obtained from the glass using a swab seeded, and counted; to FIG. 8, presenting the media bacterial load. Media bacterial load was measured after 3, 11 and 13 days of incubation; the media was sampled, seeded, incubated and counted; and to the photo in FIG. 9, displaying the media turbidity—Representative growing media picture after 3 days of incubation.

The antifouling properties of G5-coating were tested using closed aerobic system with E. coli. The measured criterion tested was the bacterial load present on the PS slide and the bacterial load in the media. Biofilm counts are presented relatively to uncoated control slide (FIG. 7). The G5 Composition was found beneficial in antifouling and it decreased the bacterial load relatively to control. Sequential results were found when the bacterial load was measured in the media (FIG. 8). A representative image of media turbidity is presented (FIG. 9). Using pH measurements we demonstrated that the antibacterial effect is not consequence of media acidity (in both treated and untreated tube pH=8-9).

It is acknowledged that throughout the entire Experimental Data section, the below terminology and annotation is applicable. Unless otherwise stated, each experiment was carried out with six types of plastic films as follows: Nafion™; 500 micron thick polyacrylamide with immobilines on polyester base pH 10; the same, at pH 9;500 micron polyacrylamide on polyester pH 5 and Control-polyester film

### Example 5

### Shelf Life Tests on Milk

The films of the present invention were tested for their effect on milk shelf life.

### Materials and Methods

Pasteurized, homogenized milk was used in order to test milk stability with the films of the present invention. In both sets of experiments the milk was LTV treated.

Test 1: Seven empty 35 mm Petri plates were filled to the top with fresh milk. Six plates were covered with the films of the present invention, so that their active side contacted the milk w/o air between them. The seventh plate was used as a control. Plates were placed on the table at room temperature for six days. Each day the pH of the plate was tested. In order to compensate for evaporation, sterile DDW was added each day. The total volume of added DDW was less then 5% of the total milk volume and therefore was not expected to influence pH dynamics. This experiment was repeated twice.

Test 2—14 day test with Nafion™: This test was performed with commercial Nafion™ as the active material (layer). Pasteurized, homogenized milk (w/o antibiotics) was used in order to test milk stability. Three empty 35 mm Petri plates were filled with fresh milk up to the top. Two were covered with Nafion™, so that active side contacted the milk w/o air between them. The third plate was used as control. Plates were placed on the table at room temperature for fourteen day. Each day pH of the plate was tested. In order to compensate for evaporation, sterile DDW was added each day. The total volume of added DDW was less then 5% of the total milk volume and therefore was not expected to influence pH dynamics.

Testing total microbial and fungal agents: This was tested on Saburo agar using the “sedimentation” method. Uncovered plates with Saburo agar were placed for 8 hours in the open. A piece of Nafion™ (10 mm×10 mm) was placed on the testing plate with active side down. Following overnight incubation at 37° C., the number of colonies was evaluated. Test and control groups were compared.

### Results

The pH results of the milk following test 1 are recorded in Table 4 herein below.

TABLE 4
pH values of milk sample

| | 1 | 2 | 3 | 4 | 5 | 6 |
|---|---|---|---|---|---|---|
| | day | day | day | day | day | day |
| Film 1 | 7.4 | 7.2 | 6.9 | 6.8 | 6.7 | 6.3 |
| Film 2 | 7.4 | 7.3 | 6.8 | 6.6 | 6.2 | 6.1 |
| Film 3 | 7.4 | 7.3 | 6.9 | 5.9 | 5.4 | 4.9 |
| Film 4 | 7.4 | 7.0 | 6.6 | 6.1 | 5.5 | 4.7 |
| Film 5 | 7.4 | 6.8 | 6.2 | 5.6 | 4.4 | 3.7 |
| Film 6. | 7.4 | 7.0 | 6.6 | 5.6 | 4.8 | 4.1 |
| Control | 7.4 | 6.9 | 6.1 | 5.4 | 4.1 | 4.0 |

The pH results of the milk (test 1, repeat experiment) are recorded in Table 5 herein below

TABLE 5
pH values of milk sample

| Day | pH |
|---|---|
| Day 0 | 8.5 |
| Day 1 | 8.7 |
| Day 2 | 8.8 |
| Day 3 | 8.7 |
| Day 4 | 8.5 |
| Day 5 | 8.6 |
| Day 6 | 8.9 |
| Day 7 | 8.5 |
| Day 8 | 8.3 |
| Day 9 | 8.5 |
| Day 10 | 8.7 |
| Day 11 | 8.8 |
| Day 12 | 8.5 |
| Day 13 | 8.5 |
| Day 14 | 8.4 |
| Day 15 | 8.5 |

The pH results of the 14 day test (test 2) are recorded in Table 6 herein below.

TABLE 6
pH values in two PSSs and a control

| | 1 | 2 | 3 | 4 | 5 | 6 | 7 |
|---|---|---|---|---|---|---|---|
| | day | day | day | day | day | day | day |
| Nafion TM 1 | 7.5 | 7.4 | 7.3 | 7.1 | 7.1 | 7 | 6.8 |
| Nafion TM 2 | 6.8 | 6.6 | 6.6 | 6.7 | 6.6 | 6.5 | 6.5 |
| Control | 7.4 | 6.7 | 6.2 | 5.1 | 4.2 | 4.1 | 4.1 |
| | 8 | 9 | 10 | 11 | 12 | 13 | 14 |
| | day | day | day | day | day | day | day |
| Nafion TM 1 | 6.8 | 6.6 | 6.6 | 6.7 | 6.6 | 6.5 | 6.5 |
| Nafion TM 2 | 4.7 | 4.6 | 4.4 | 4.5 | 4.4 | 4.4 | 4.3 |
| Control | 4.2 | 4.2 | 4.1 | 4.1 | 4.2 | 4.1 | 4.1 |

The results from testing total microbial and fungal agents are recorded in Table 7 herein below.

TABLE 7
Total microbial and fungi agents

| | Total microbial and fungi agents | | |
|---|---|---|---|
| | (colonies) | | |
| No | First | Second | Control |
| 1 | 2 | 1 | 14 |
| 2 | 2 | 2 | 31 |
| 3 | 3 | 3 | 24 |
| 4 | 0 | 6 | 25 |
| 5 | 4 | 5 | 16 |
| 6 | 0 | 2 | 20 |
| 7 | 2 | 5 | 19 |
| 8 | 3 | 2 | 13 |
| 9 | 2 | 2 | 37 |
| 10 | 1 | 3 | 25 |

### Test 3: Milk Test

### Materials and Methods

500 ml of UHT milk had been inoculated with Staphylococcus caseolyticus (in a final concentration of 1×10⁷ CFU/ml) and kept in a two glass vessels: one with BioActivity™ coating (Nafion™-coated glass vessel) and one without at room temperature. On time 0 (time of inoculation) and 3, 7, 14 and 17 days of incubation at room temperature, UHT milk from both vessels had been sampled and 10-fold dilutions were plated on TSA media and the number of colony forming units of S. caseolyticus per ml of milk was calculated.

### Results

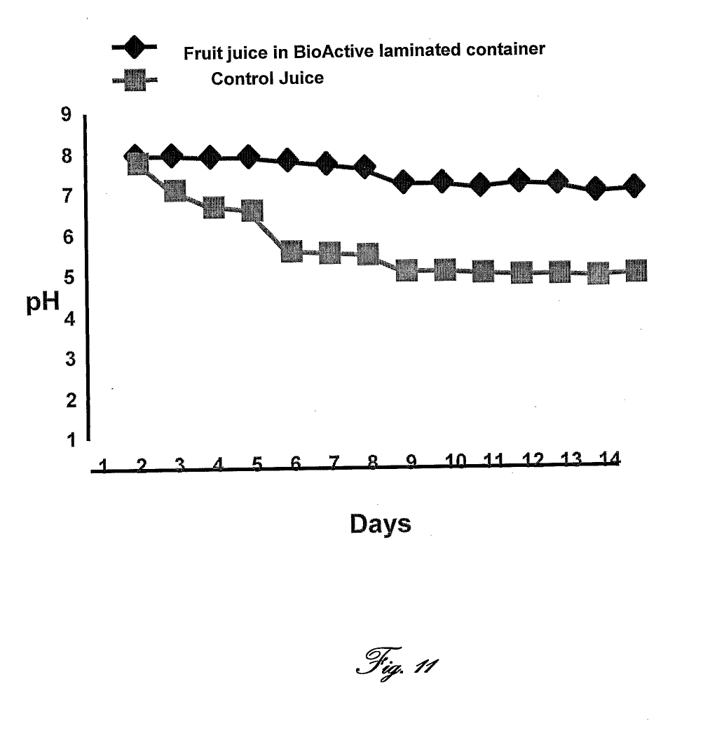

Reference is now made to FIG. 10, illustrating the effect of BioActivity™ coating of glass vessels on S. caseolyticus-inoculated UHT milk.

After 3 days, the milk kept in the BioActivity™ coated vessel was in the same condition as in time 0. The milk in the control vessel without the coating was spoiled (pieces of solids could have been seen and strong smell was in the air). The number of S. caseolyticus CFU/ml reached the level of 10¹⁴ in the control whereas in the BioAvtive treatment it remained stable at the initial level (1×10⁷ CFU/ml) (FIG. 10)

After 7 days the milk in the control was totally degraded and spoiled and phase separated while in the BioAvtive treatment the milk was in the same condition as in the first day. S. caseolyticus counts in the control reached the level of 10¹⁵ CFU/ml and in the BioAvtive treatment it remained at the initial level of 1×10⁷ CFU/ml.

This picture remained until the end of the experiment after 14 and 17 days. (FIG. 11)

### Example 6

### Fruit Juice Stability

### Materials and Methods

Pasteurized fruit juice (named “tropic”) was used in order to test the effect of BioActivity™ laminates (i.e., laminates provided by means and methods of the present invention) on fruit juice stability with. Six empty 35 mm Petri dishes were filled with fresh fruit juice up to the top. Five of which were covered with BioActivity™ laminates, so that the active side contacted the fruit juices w/o air between them. The sixth dish was used as control. The Petri dishes were placed on the table at room temperature for 14 days. In order to compensate of evaporation, sterile DDW were added each day to a total volume of less then 5% of the total fruit juices volume (in order not to influence on pH dynamics). The pH value of the juice was measured each day.

### Results

Reference is now made to FIG. 11 displaying the pH dynamics of fruit juice stored in BioActivity™ laminated containers and in control container for 14 days at room temperature. The pHs in all BioActivity™ laminates treated juice samples remain stable throughout the experiment whereas in the control sample, the pH gradually declined and reached the value of 5.2 by the 14^{th} day (table 7 and FIG. 11)

TABLE 7
Fruit juice experiment

| | 1 | 2 | 3 | 4 | 5 | 6 | 7 |
|---|---|---|---|---|---|---|---|
| | day | day | day | day | day | day | day |
| First layer | 7.86 | 7.78 | 7.78 | 7.74 | 7.63 | 7.6 | 7.53 |
| Second layer | 7.72 | 7.69 | 7.64 | 7.55 | 7.55 | 7.46 | 7.36 |
| Third layer | 7.97 | 7.84 | 7.86 | 7.85 | 7.68 | 7.71 | 7.61 |
| Fourth layer | 8.14 | 8.14 | 8.10 | 8.06 | 8.00 | 7.91 | 7.82 |
| Fifth layer | 7.96 | 7.96 | 7.85 | 7.85 | 7.75 | 7.67 | 7.70 |
| Control | 8.11 | 7.35 | 6.92 | 6.83 | 5.81 | 5.77 | 5.71 |
| | 8 | 9 | 10 | 11 | 12 | 13 | 14 |
| | day | day | day | day | day | day | day |
| First layer | 7.14 | 7.10 | 6.93 | 6.98 | 6.89 | 6.85 | 6.87 |
| Second layer | 7.01 | 6.96 | 6.87 | 6.92 | 6.9 | 6.83 | 6.67 |
| Third layer | 7.26 | 7.29 | 7.28 | 7.18 | 7.10 | 7.15 | 6.97 |
| Fourth layer | 7.43 | 7.41 | 7.35 | 7.42 | 7.37 | 7.19 | 7.26 |
| Fifth layer | 7.21 | 7.21 | 7.10 | 7.10 | 7.00 | 6.92 | 6.95 |
| Control | 5.29 | 5.31 | 5.26 | 5.22 | 5.23 | 5.16 | 5.20 |

### Example 7

### Control of Salmonella Contamination on Fresh Eggs

### Material and Methods

Six fresh eggs were placed into solution with Salmonella typhimurium (˜10⁶/ml) for 15 min. Then eggs were tightly covered with layers, each egg separately in its own cover. Eggs were then stored in refrigerator 4° C. for one week. After fortnight period eggs were washed with 20 ml of sterile DDW. The resulted washing water was centrifuged (3000 RPM/10 min) and sediments were spread on Petri dishes with McConcy selective media. Suspicious colonies were tested by agglutination test with polyclonal anti-Salmonella serum.

### Results

No specific reaction (that indicates Salmonella contamination) could be observed on all five treated eggs. On the other hand, specimens took from control eggs demonstrated specific agglutination that points on Salmonella contamination (Table 8).

TABLE 8
Eggs samples

| | |
|---|---|
| Laminate 1 | Negative |
| Laminate 2 | Negative |
| Laminate 3 | Negative |
| Laminate 4 | Negative |
| Laminate 5 | Negative |
| Control | Positive |

### Example 8

### Shelf Life Tests on Beef Meat

### Test 1 Material and Methods

Fresh beef flesh was cut into small (˜1 cm) pieces. Each piece was places into a 35 mm Petri dish and incubated for one week at 23±2° C. At the end of the incubation period each piece was homogenized and analyzed for coliforms contamination on ENDO media.

### Results

The number of colony forming units (CFU) per gr in all BioActivity™ materials treated samples was less then 10³ (considered as a Fresh meat). Control sample contained more then 10⁶ (Table 9)

TABLE 9
Beef Meat samples

| | 7^{th} day |
|---|---|
| First layer | 6.7 × 10² |
| Second layer | 8.2 × 10² |
| Third layer | 5.2 × 10² |
| Fourth layer | 7.0 × 10² |
| Fifth layer | 6.3 × 10² |
| Control | >10⁶ |

### Test 2 Material and Methods

Fresh beef flesh was cut into small ˜1 cm pieces. Each piece was places into six 35 mm Petri plates for one week. After seven days each piece was homogenized and analyzed microbiologically for coli-forming flora on ENDO media. The final result is a number of colony forming units. (For fresh meat less then 1000 CFU/gr)

### Results

The numbers of colony forming units (CFU) per gr. in all BioActivity™ materials treated samples were within the acceptable standard (considered as a Fresh meat) except for one outlier which was slightly above the standard (1.3×10³ CFU/gr). Control sample on the other hand, contained more then 10⁶ (Table 10)

TABLE 10
Fresh meat samples

| | 7^{th} day |
|---|---|
| First layer | 8.8 × 10² |
| Second layer | 8.2 × 10² |
| Third layer | 1.0 × 10³ |
| Fourth layer | 9.1 × 10² |
| Fifth layer | 1.3 × 10³ |
| Control | >10⁶ |

### Test 3

### Chopped Meat

### Material and Methods

Fresh beef meat was cut into small pieces (˜0.1 cm each). Each piece (˜5 g each) was places into a 35 mm Petri dish and incubated at 23±2° C. for one week. At the end of the incubation period, each piece of the chopped meat was homogenized and analyzed for coliforms contamination on ENDO media. The final result is the number of colony forming units (CFU) per gr. of chopped meat (the standard for fresh meat is less then 10³ CFU/gr.)

### Results

The number of colony forming units (CFU) per gr. in all BioActivity™ materials treated samples was less then 10³ (considered as a Fresh meat). Control sample contained more then 10⁶ (Table 11)

TABLE 11
Chopped Meat saples

| | 7^{th} day |
|---|---|
| First layer | 7.2 × 10² |
| Second layer | 8.8 × 10² |
| Third layer | 6.3 × 10² |
| Fourth layer | 7.7 × 10² |
| Fifth layer | 9.0 × 10² |
| Control | >10⁶ |

### Example 9

### Vegetables

### Test 1 Cherry Tomato Test

### Materials and Methods

Cherry tomato were cut to half and placed into a 35 mm Petri dish for one and incubated at 23±2° C. for one week. At the end of the incubation period, each piece was homogenized and analyzed for total microbial count on Saburo media. The final result is a number of colony forming units per gr. of fruit material. (The standard is less then 10³ CFU/gr)

### Results

In all BioActivity™ laminate treated samples, total bacterial counts were less then the standard (at the range of 3.7 to 8.9×10² CFU/gr) whereas in the control, the counts were above the standard (Table 12)

TABLE 12
Cherry tomato samples

| | 7^{th} day |
|---|---|
| First layer | 4.8 × 10² |
| Second layer | 6.7 × 10² |
| Third layer | 5.7 × 10² |
| Fourth layer | 3.7 × 10² |
| Fifth layer | 8.9 × 10² |
| Control | 1.7 × 10³ |

### Test 2 Cucumber Test

Fresh cucumbers were cut into small ˜1 cm pieces. Each piece was places into a 35 mm Petri dish and incubated at 23±2° C. for one week. At the end of the incubation period each piece was homogenized and analyzed for coliforms contamination on ENDO media. The final result is the number of colony forming units (CFU) per gr. material (the standard for fresh vegetables is less then 10³ CFU/gr)

### Results

In all BioActivity™ laminate treated samples, total bacterial counts were less then the standard (at the range of 3.7 to 5.2 CFU/gr) whereas in the control, the counts were high above the standard (Table 13)

TABLE 13
Cucumber test

| | 7^{th} day |
|---|---|
| First layer | 3.7 × 10² |
| Second layer | 4.1 × 10² |
| Third layer | 3.8 × 10² |
| Fourth layer | 4/9 × 10² |
| Fifth layer | 5.2 × 10² |
| Control | >10⁶ |

### Example 10

### Fruit Tests

### Material and Methods

Test 1 Fresh cherry fruits were places into 35 mm Petri dishes and incubated at 23±2° C. for one week. At the end of the incubation period, each berry was homogenized and analyzed for total microbial count on Saburo media. The final result is a number of colony forming units (CFU) per gr. material. (The standard is less then 10³ CFU/gr).

### Results

In all BioActivity™ laminate treated samples, total bacterial counts were less then the standard (at the range of 1.2 to 5.4×10² CFU/gr.) whereas in the control the counts were above the standard (Table 14)

TABLE 14
Fruit samples

| | 7^{th} day |
|---|---|
| First layer | 1.2 × 10² |
| Second layer | 3.5 × 10² |
| Third layer | 4.5 × 10² |
| Fourth layer | 2.7 × 10² |
| Fifth layer | 5.4 × 10² |
| Control | 1.2 × 10³ |

Test 2 Pieces of fresh of Loquat medlar fruits (Eriobotrya japonica) were places into 35 mm Petri dishes and incubated at 23±2° C. for one week. At the end of the incubation period each piece was homogenized and analyzed for total microbial count on Saburo media. The final result is the number of colony forming units (CFU) per gr. material (The standard is less then 10³ CFU/gr)

### Results

In all BioActivity™ laminate treated samples, total bacterial counts were less then the standard (at the range of 3.2 to 4.5×10² CFU/gr.) whereas in the control the counts were much higher and very close to the permitted standard (Table 15).

TABLE 15
Eriobotrya japonica samples

| | 7^{th} day |
|---|---|
| First layer | 3.9 × 10² |
| Second layer | 3.2 × 10² |
| Third layer | 4.5 × 10² |
| Fourth layer | 4.1 × 10² |
| Fifth layer | 3.5 × 10² |
| Control | 9.1 × 10² |

Test 3 Fresh peach pieces were places into 35 mm Petri dishes for and incubated at 23±2° C. for one week. After seven days each piece was homogenized and analyzed microbiologically for total microbial counts on Saburo media. The final result is the number of colony forming units (CFU) per gr. material (The standard is less then 10³ CFU/gr)

### Results

In all BioActivity™ laminate treated samples, total bacterial counts were less then the standard (at the range of 2.8 to 6.5×10² CFU/gr.) whereas in the control the counts were above the standard (Table 16)

TABLE 16
Fresh peach samples

| | 7^{th} day |
|---|---|
| First layer | 4.8 × 10² |
| Second layer | 4.6 × 10² |
| Third layer | 3.6 × 10² |
| Fourth layer | 2.8 × 10² |
| Fifth layer | 6.5 × 10² |
| Control | 1.4 × 10³ |

### Example 11

### Example of Coated Jars with Shampoo Solution

The purpose of this experiment is to evaluate antibacterial properties of coating and prove negligible migration of the active component from a coating. Bioactive silicone based resin was prepared by copolymerization of the following components: 15% 2-phenyl-5-benzimidazole-sulfonic acid (Sigma 437166-25 ml); 80% Siloprene LSR 2060 (GE); 5% plastificator RE-AS-2001 (Sigma 659401-25 ml); 1 g of the mixture was spread in walls of glass jars (thickness 1 g/10 cm**2) and polymerized at 200 deg C. for 3 hours.

These coated jars and control jars without coating were used to test antibacterial activity of a solution of a cosmetic shampoo without preservatives. The solution of S. aureus bacteria input: 40.000 cfu/ml was used for inoculation of the shampoo solution. 5 ml of TSB+S. aureus bacteria were added into a jar.

After intervals of 24 hours all samples were sampled and decimal diluted spread on TSA plates. After 24 hours of incubation at 30° C. colonies were counted. The results are presented in the following tables.

### Results

TABLE 17
Antibacterial activity of coated jars without shampoo

| Jars | cfu/ml |
|---|---|
| EL-18 febr. #1 | 0 |
| Control (w/o coating) | >10¹¹ |

TABLE 18
Antibacterial activity of coated jars in the presence of 10% Shampoo
(after 24 hrs incubation)

| Jars | cfu/ml |
|---|---|
| EL-18 febr. #2 | 3 × 10⁴ |
| EL-18 febr. #3 | 3.3 × 10⁴ |
| Control (w/o coating) | 6 × 10⁶ |

Test on coated jars with Candida albicans

TABLE 19
Test microorganisms
Test Microorganisms: Candida albicans (ATCC: 0231) 1.3 × 10⁴ CFU/ml
Results:

| Sample | | CFU/Sample | Log | pH |
|---|---|---|---|---|
| Expermient | Time “0” | 1.1 × 10³ | 3.04 | 7.60 |
| 2 in Jars | Control after “24 h” | 4.0 × 10³ | 5.6 | 6.94 |
| | Samples 3A after “24 h” | <1 | 0 | 7.77 |

TABLE 20
Antibacterial activity of coated jars in the presence of 10% Shampoo
(after 72 hrs incubation)

| Jars | cfu/ml |
|---|---|
| EL-18 febr. #2 | 0 |
| EL-18 febr. #3 | 0 |
| Control (w/o coating) | 1.2 × 10⁹ |

TABLE 21
Antibacterial activity of coated jars in the presence of 10% Shampoo
(after 168 hrs incubation)

| Jars | cfu/ml |
|---|---|
| EL-18 febr. #2 | 0 |
| EL-18 febr. #3 | 0 |
| Control (w/o coating) | 4.3 × 10¹¹ |

pH values were equal to 7 in Jars EL-18 febr #1-4.

For leaching experiment, 5 ml of sterile water were added to the EL-18 febr. #4 and control jars. Incubation was performed 48 hrs at 30° C. K, Na, S and Si were determined by ICP method.

TABLE 22
ICP analysis

| Samples | Elements | mg/l |
|---|---|---|
| Control (#1) | Na | 1.49 |
| (pH 7) | K | 0.056 |
| | S | 0.66 |
| | Si | 0.13 |
| Silicone coating | Na | 0.81 |
| (pH 7) | K | 0.01 |
| | S | 0.07 |
| | Si | 0.009 |

The results show negligible release of materials from the coating

1-35. (canceled)

36. Biocidic packaging effective for killing cells, said packaging comprising at least one charged polymer, said at least one charged polymer characterized, when in contact with a water-containing environment, as:
a. carrying strongly acid and/or strongly basic functional groups;
b. having a pH of less than about 4.5 or greater than about 8.0;
c. capable of generating an electrical potential within the confined volume of said cell sufficient to disrupt effectively the pH homeostasis and/or electrical balance within said confined volume of said cell; and,
d. being in a form chosen from the group consisting of (i) H⁺ and (ii) OH⁻;
wherein said charged polymer is adapted to preserve the pH of said cell's environment.

37. The packaging of claim 36, further characterized, when said groups are accessible to water, as having a buffering capacity of about 20 to about 100 mM H⁺/L/pH unit.

38. The packaging of claim 36, further characterized, when said groups are accessible to water, by at least one characteristic chosen from the group consisting of (a) sufficiently water-insoluble such that at least 99.9% remains undissolved at equilibrium; (b) sufficiently resistant to leaching such that the total concentration of material leached from said composition of matter into said water-containing environment does not exceed 1 ppm; (c) sufficiently inert such that at least one parameter of said water-containing environment chosen from the group consisting of (i) concentration of at least one predetermined water-soluble substance; (ii) particle size distribution; (iii) rheology; (iv) toxicity; (v) color; (vi) taste; (vii) smell; and (viii) texture remains unaffected according to preset conditions, said conditions adapted for and appropriate to said particular environment.

39. The packaging of claim 36, further comprising at least one polymer chosen from the group consisting of (a) polyvinyl alcohol; (b) polystyrene sulfonate; and (c) polypropylene polystyrene-divinylbenzene.

40. The packaging of claim 39, wherein at said at least one polymer contains at least one functional group chosen from the group consisting of SO₃H and H₂N(CH₃).

41. The packaging of claim 1, further comprising hydrophilic additives chosen from the group consisting of proton conductive materials (PCMs) and hydrophilic polymers (HPs); further wherein said PCMs and HPs are chosen from the group consisting of (a) sulfonated tetrafluoroethylene copolymers; (b) sulfonated materials chosen from the group consisting of silica, polythion-ether sulfone (SPTES), styrene-ethylene-butylene-styrene (S-SEBS), polyether-ether-ketone (PEEK), poly(arylene-ether-sulfone) (PSU), polyvinylidene fluoride (PVDF)-grafted styrene, polybenzimidazole (PBI), and polyphosphazene; and (c) proton-exchange membranes made by casting a polystyrene sulfonate (PSSnate) solution with suspended micron-sized particles of cross-linked PSSnate ion exchange resin.

42. The packaging of claim 36, comprising two or more charged polymers chosen from the group consisting of two-dimensional charged polymers and three-dimensional (3D) charged polymers, each of which of said charged polymers comprises materials containing cationic and/or anionic groups capable of dissociation and spatially organized in a manner adapted to preserve the pH of said water-containing environment according to preset conditions; said spatial organization chosen from the group consisting of (a) interlacing; (b) overlapping; (c) conjugating; (d) homogeneously mixing; (e) heterogeneously mixing; and (f) tiling.

43. The packaging of claim 36, further comprising at least one proton-permeable surface with a given functionality, said surface layers with said charged polymer.

44. The packaging of claim 36, further comprising a surface with a given functionality and at least one external proton-permeable layer, each of which of said at least one external proton-permeable layers is disposed on at least a portion of said surface.

45. The packaging of claim 36, comprising at least one charged polymer and at least one barrier adapted to prevent heavy ion diffusion.

46. The packaging of claim 36, wherein said packaging is in the form of a continuous barrier, said barrier selected from the group consisting of (a) 2D pads; (b) 3D pads; (c) sponges; (d) nonwoven webs; (e) membranes; (f) filters; (g) meshes; (h) nets; (i) sheet-like members; (j) any combination of the above.

47. The packaging of claim 36, wherein said packaging is in the form of an insert of dimensions adapted to allow mounting within an article of manufacture of predetermined dimensions, said mounting chosen from the group consisting of reversible mounting and permanent accommodation.

48. The packaging of claim 47, wherein said insert is in a form chosen from (a) membrane; (b) wrap; (c) separating sheets; (d) foil; (e) rod; (f) mesh; (g) spheres; (h) beads; (i) float; and (j) ring.

49. The packaging of claim 36, wherein said charged polymer is incorporated into and/or comprises and/or coats at least part of a sealing device chosen from the group consisting of (a) cap; (b) lid; (c) stopper; (d) cork; and (e) seal.

50. The packaging of claim 36, wherein said packaging is in a form chosen from the group consisting of (a) powder; (b) gel; (c) suspension; (d) spray; (e) resin; (f) coating; (g) film; (h) sheet; (i) bead; (j) particle; (k) microparticle; (l) nanoparticle; (m) fiber; (n) thread.

51. The packaging of claim 36, further characterized by at least one of the following:
a. capacity for absorbing or releasing protons capable of regeneration;
b. buffering capacity capable of regeneration; and
c. proton conductivity capable of regeneration.

52. A method for increasing the rate of death of living cells and/or decreasing the rate of reproduction of living cells within a water containing-environment, comprising the steps of:
a. providing packaging comprising at least one charged polymer, said at least one charged polymer characterized, when in contact with said water-containing environment, as:
i. carrying strongly acid and/or strongly basic functional groups;
ii. having a pH of less than about 4.5 or greater than about 8.0;
iii. capable of generating an electrical potential within the confined volume of said cell sufficient to disrupt effectively the pH homeostasis and/or electrical balance within said confined volume of said cell; and,
iv. being in a form chosen from the group consisting of (i) H⁺ and (ii) OH⁻; and,
b. placing said packaging in contact with said water-containing environment.

53. The method of claim 52, wherein said step (a) further comprises the step of providing said charged polymer with predetermined water permeability, proton conductivity, and/or wetting characteristics, and further wherein said water permeability, proton conductivity, and/or wetting characteristics are provided by at least one substance selected from the group consisting of proton conductive materials (PCMs) and hydrophilic polymers (HPs).

54. The method of claim 53, wherein said step of providing said charged polymer with predetermined water permeability, proton conductivity, and/or wetting characteristics, and further wherein said water permeability, proton conductivity, and/or wetting characteristics are provided by at least one substance selected from the group consisting of proton conductive materials (PCMs) and hydrophilic polymers (HPs) further comprises a step of choosing said PCMs and HPs from the group consisting of (a) sulfonated tetrafluoroethylene copolymers; (b) sulfonated materials chosen from the group consisting of silica, polythion-ether sulfone (SPTES), styrene-ethylene-butylene-styrene (S-SEBS), polyether-ether-ketone (PEEK), poly(arylene-ether-sulfone) (PSU), polyvinylidene fluoride (PVDF)-grafted styrene, polybenzimidazole (PBI), and polyphosphazene; (c) proton-exchange membranes made by casting a polystyrene sulfonate (PSSnate) solution with suspended micron-sized particles of cross-linked PSSnate ion exchange resin; and derivatives thereof.

55. The method of claim 52, further comprising a step of providing at least one polymer chosen from the group consisting of (a) polyvinyl alcohol; (b) polystyrene sulfonate; and (c) polypropylene polystyrene-divinylbenzene.

56. The method of claim 52, wherein said step of providing at least one polymer further comprises a step of providing at least one polymer that contains at least one functional group chosen from the group consisting of SO₃H and H₂N(CH₃).

57. The method of claim 52, further comprising a step of providing two or more charged polymers chosen from the group consisting of two-dimensional charged polymers and three-dimensional (3D) charged polymers, each of which of said charged polymers comprises materials containing cationic and/or anionic groups capable of dissociation and spatially organized in a manner adapted to preserve the pH of said water-containing environment according to preset conditions; said spatial organization chosen from the group consisting of (a) interlacing; (b) overlapping; (c) conjugating; (d) homogeneously mixing; (e) heterogeneously mixing; and (f) tiling.

58. The method of claim 57, further comprising a step of spatially organizing each of said functional groups in a manner selected from (a) interlacing; (b) overlapping; (c) conjugating; (d) homogeneously mixing; (e) heterogeneously mixing; and (f) any combination of the above.

59. The method of claim 52, further comprising an additional step of providing said charged polymer with an ionomeric barrier layer comprising a sulfonated tetrafluoroethylene copolymer, said barrier adapted to avoid heavy ion diffusion.

60. A method of production of a biocidic packaging, comprising the steps of:
a. providing at least one charged polymer, said at least one charged polymer characterized, when in contact with said water-containing environment, as:
i. carrying strongly acid and/or strongly basic functional groups;
ii. having a pH of less than about 4.5 or greater than about 8.0;
iii. capable of generating an electrical potential within the confined volume of said cell sufficient to disrupt effectively the pH homeostasis and/or electrical balance within said confined volume of said cell; and,
iv. being in a form chosen from the group consisting of (i) H⁺ and (ii) OH⁻; and,
b. adapting said charged polymer to a form chosen from the group consisting of (a) powder; (b) gel; (c) suspension; (d) resin; (e) coating; (f) film; (g) sheet; (h) bead; (i) particle; (j) microparticle; (k) nanoparticle; (l) fiber; (m) thread; (n) shape.

61. The method of claim 60, wherein said step of providing at least one electrolyte charged polymer characterized, when in contact with said water-containing environment, by at least one characteristic chosen from the group consisting of (a) sufficiently water-insoluble such that at least 99.9% remains undissolved at equilibrium; (b) sufficiently resistant to leaching such that the total concentration of material leached from said composition of matter into said water-containing environment does not exceed 1 ppm; (c) sufficiently inert such that at least one parameter of said water-containing environment chosen from the group consisting of (i) concentration of at least one predetermined water-soluble substance; (ii) particle size distribution; (iii) rheology; (iv) toxicity; (v) color; (vi) taste; (vii) smell; and (viii) texture remains unaffected according to preset conditions, said conditions adapted for and appropriate to said particular environment.

62. The method of claim 60, wherein said step of providing at least one electrolyte further comprises the step of providing a charged polymer characterized, when in contact with said water-containing environment, as being sufficiently inert such that the toxicity said water-containing environment as defined by at least one parameter chosen from the group consisting of (a) LD₅₀ and (b) ICT₅₀ remains unaffected according to preset conditions, said conditions adapted for and appropriate to said particular environment.

63. The method of claim 60, further comprising the steps of:
c. providing a substance characterized by at least one surface; and
d. locating said charged polymer on at least one surface of said substance.

64. The method of claim 60, further comprising steps of:
c. providing at least one external proton-permeable surface with a predetermined functionality; and
d. layering at least a portion of said proton-permeable surface with at least one of said charged polymer.

65. The method of claim 60, wherein said step of providing at least one polymer further comprises a step of providing at least one polymer chosen from the group consisting of (a) polyvinyl alcohol; (b) polystyrene sulfonate; and (c) polypropylene polystyrene-divinylbenzene.

66. The method of claim 60, wherein said step of providing at least one polymer that contains at least one functional group chosen from the group consisting of SO₃H and H₂N(CH₃).

67. A method for regenerating the biocidic properties of packaging as defined in claim 36, said method comprising at least one step chosen from the group consisting of (a) regenerating said packaging's proton absorbing and/or releasing capacity; (b) regenerating said packaging's buffering capacity; and (c) regenerating the proton conductivity of said packaging.
